(19) United States
(12) Patent Application Publication
(10) Pub. No.: US 2016/0374853 A1
(43) Pub. Date: Dec. 29, 2016

(54) METHODS OF TRANSCUTANEOUS HEAT TRANSFER, AND DEVICES AND SYSTEMS FOR USE IN THE SAME

(71) Applicant: THE BOARD OF TRUSTEES OF THE LELAND STANFORD JUNIOR UNIVERSITY, Stanford, CA (US)

(72) Inventors: Dennis A. Grahn, Palo Alto, CA (US); H. Craig Heller, Stanford, CA (US)

(21) Appl. No.: 15/113,788

(22) PCT Filed: Feb. 2, 2015
(86) PCT No.: PCT/US15/14072

§ 371 Date: Jul. 22, 2016

Related U.S. Application Data
(60) Provisional application No. 61/935,196, filed on Feb. 3, 2014.

(51) Int. Cl. A61F 7/08; A61F 7/00

(52) U.S. Cl. CPC A61F 7/08; A61F 7/007; A61F 2007/0036; A61F 2007/0047; A61F 2007/0054; A61F 2007/0075; A61F 2007/0078

(57) ABSTRACT

Methods for transcutaneous heat transfer are provided. Aspects of the methods include stably associating a glabrous skin surface of a mammal with a thermoregulatory device configured to be associated with only a portion of a limb of the mammal and transferring heat through the glabrous skin surface without application of negative pressure. Also provided are devices, systems and kits that fmd use practicing the methods. The methods and compositions described herein find use in a variety of different applications, including maintaining normothermia, e.g., in thermoregulatory compromised individuals.

### CROSS REFERENCE To RELATED APPLICATIONS

Pursuant to 35 U.S.C. §119 (e), this application claims priority to the filing date of the U.S. Provisional Patent Application Ser. No. 61/935,196, filed Feb. 3, 2014; the disclosure of which is herein incorporated by reference.

### INTRODUCTION

The body temperature of mammals is normally controlled by an internal autonomic regulatory system referred to herein as the thermoregulatory system. One important effector in this system is controlled by blood flow to specialized skin areas of the body at non-hairy skin surfaces (i.e., at the palms, soles of the feet, cheeks/nose regions). Subcutaneous to these areas, there are unique anatomical vascular structures called venous plexuses. These structures serve to deliver large volumes of blood adjacent the skin surface. By this delivery of blood, significant heat transfer is enabled for the maintenance of internal organs within a functional temperature range. Blood is permitted to pass through the venous plexuses “radiator” structures by way of arterio venous anastamoses, or AVAs that gate or control the arterial input side of the venous plexuses. Thus, the AVAs serve an integral part of the heat transfer system, providing thermoregulatory control. Together, the AVAs and venous plexuses make up a body's relevant heat exchange vasculature.

Typically, when body and or environmental temperatures are high, dilation of certain blood vessels favors high blood flow to the noted heat exchange surfaces, thus increasing heat loss to the environment and reduction in the deep body core region temperature. As environmental and/or body temperatures fall, vasoconstriction reduces blood flow to these surfaces and minimizes heat loss to the environment.

### SUMMARY

Methods for transcutaneous heat transfer are provided. Aspects of the methods include stably associating a glabrous skin surface of a mammal with a thermoregulatory device configured to be associated with only a portion of a limb of the mammal and transferring heat through the glabrous skin surface without application of negative pressure. Also provided are devices, systems and kits that find use practicing the methods. The methods and compositions described herein find use in a variety of different applications, including maintaining normothermia, e.g., in thermoregulatory compromised individuals.

### BRIEF DESCRIPTION OF THE FIGURES

FIG. 1 provides a partial cross-sectional side view of a device according to an embodiment of the invention.

FIG. 2 provides a partial cross-sectional side view of a device according to according to an embodiment of the invention, where the device is shown in heat transfer relationship with a portion of a mammal.

FIG. 3 provides a top view of a device according to an embodiment of the invention.

FIG. 4 provides a top view of embodiments of devices according to an embodiment of the invention.

FIG. 5 provides a partial cross-sectional side view of a device according to an embodiment of the invention.

FIG. 6 provides a schematic of an embodiment of a system.

FIG. 7 provides graphical results showing the core temperature of individuals during exposure to surgical levels of anesthesia with countermeasures to protect core temperature compared to no temperature management treatment. Patients subjected to temperature countermeasures were undergoing Transjugular intrahepatic portosystemic shunt (TIPS) surgery. Tes—Esophageal temperature. Red diamonds—heat delivered via forced warm air to the torso and leg regions (Standard of practice for perioperative temperature management). Blue squares—application of a regulated heat source to the glabrous skin regions of the hands and feet. Black diamonds—no treatment.

FIG. 8. provides a picture of a subject in a personal protective suit as described in Example II, below.

FIG. 9 provides a picture of a palmer cooling device used in conjunction with a personal protective suit, as described in Example II, below.

FIG. 10 graphically illustrates the core (esophageal) temperature (Tes) of an individual during mild exercise in a hot environment while clad in a DuPont Tychem QC biohazard personal protective suit, as shown in FIG. 8. The subject was walking at 2 miles per hour on a flat surface. Ambient temperature was 41° C. Open circles: no treatment. Closed symbols: cool water circulated over the palmar skin of 2 hands using the device shown in FIG. 9.

### DETAILED DESCRIPTION

Methods for transcutaneous heat transfer are provided. Aspects of the methods include stably associating a glabrous skin surface of a mammal with a thermoregulatory device configured to be associated with only a portion of a limb of the mammal and transferring heat through the glabrous skin surface without application of negative pressure. Also provided are devices, systems and kits that find use practicing the methods. The methods and compositions described herein find use in a variety of different applications, including maintaining normothermia, e.g., in thermoregulatory compromised individuals.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, embodiments of methods of the invention are described first in greater detail, followed by a description of devices and systems that find use in practicing the methods in accordance with the invention, as well as a review of certain applications in which the invention finds use.

### Methods

As summarized above, aspects of the invention include methods of transcutaneous heat transfer. By transcutaneous heat transfer is meant moving heat across a skin surface of a mammal. The heat transfer direction, i.e., the direction of heat transfer, may be into or out of the mammal. As such, methods of the invention include transferring heat across a skin surface into a region of a mammal, such as the circulatory system of the mammal. Methods of invention also include transferring heat across a skin surface out of a region of a mammal, e.g., from the circulatory system of the mammal to a location outside of the mammal.

Aspects of the methods include transferring heat through one or more glabrous skin surfaces of the mammal. By glabrous surface is meant a skin surface that is characterized by an absence of hair follicles and the presence of unique subcutaneuous vascular structures that enable a large volume of the circulating blood to flow directly below the skin surface. In contrast, non-glabrous skin is characterized by the presence of hair follicles and the absence of subcutaneous vascular structures. Glabrous skin surfaces of interest include palms, soles, non-hairy surfaces of the face and head, and portions of the ears, e.g., ear pinnae. In some embodiments of the methods, the methods include transferring heat through and/or via, a heat exchange surface, e.g., a sole and/or palm, of a portion of a mammal, e.g., a hand and/or foot. In certain embodiments, the methods include introducing heat into, e.g., heating, and/or extracting heat from, e.g., cooling, the body core of a mammal. The introduction or extraction of such heat may be, for example, through a heat exchange surface of a mammal. As such, in some instances the methods include transferring heat through a glabrous skin surface into the circulatory system of the mammal. In other instances, the methods include transferring heat through a glabrous skin surface from or out of the circulatory system of the mammal.

In practicing methods according to embodiments of the invention, a thermoregulatory device is stably associated with a target glabrous skin surface of the mammal in heat transfer relationship. As such, methods of the invention may include stably associating a heat exchange surface of a portion of a mammal with a thermoregulatory device. Such a portion of a mammal may include one or more limbs, such as arms and/or legs and/or portions thereof, such as one or more hands and/or feet. By “stably associating” and/or “stably associated” is meant placing in a contacting orientation, e.g., a contacting orientation wherein the surface area of the touching portions are maximized, and/or affixing to, e.g., affixing to with an attachment element, such that the disclosed methods, systems, and devices may be effectively employed. Aspects of the methods include stably associating a heat exchange surface of a portion of a mammal with a thermoregulatory device or a portion thereof, such as a heat exchange element, or a portion thereof, e.g., as described below. Upon stable associate of the heat exchange surface, such as glabrous skin surface, with the thermoregulatory device, the two components have little, if any, movement relative to each other, at least during the heat transfer process.

In certain embodiments of the subject methods, a portion of a mammal is stably associated with a thermoregulatory device, and in operative relationship (e.g., heat transfer relationship) with the heat exchange element thereof for a period of time sufficient for the desired heat transfer to occur. While the period of time of a given protocol may vary, in some instances the portion of the mammal and thermoregulatory device are stably associated with each other for a period of time ranging, for example, from 0.1 min to 1500 min, such as 1 min to 750 min, including 5 min to 180 min. Stable association may be maintained for a period of time sufficient for the desired effect, e.g., amount of heat transfer, to occur. As such, contact may be maintained for 1 min or more, such as 2 min or more, or 3 min or more, where contact may be maintained for 10 hr or longer (e.g., for the duration of a surgical procedure). In some embodiments, contact is maintained for 12 hr or less, such as 10 hours or less, including 6 hours or less.

Thermoregulatory devices that may be employed with the subject methods include each of the thermoregulatory device embodiments described herein. In some instances, thermoregulatory devices include one or more, e.g., two, three, four, etc., heat exchange elements. Heat exchange elements, in some aspects, are heating and/or cooling elements. A heating element is an element that is configured to transfer heat through a glabrous surface into a body of a mammal. A cooling element is an element that is configured to transfer heat from the body of a mammal through the glabrous skin surface into the cooling element. Any convenient heating and cooling heat exchange elements may be employed.

In some instances, the heat exchange element is a thermoelectric heat exchange element. In various embodiments, heat exchange elements include one or more thermoelectric heaters and/or coolers, e.g., an electrical heater (such as a resistive coil) and/or cooler (such as Peltier device). Heat exchange elements, in various aspects where they are thermoelectric devices, are not configured to receive fluid therein and/or therethrough and/or, in some embodiments, are not configured to heat and/or cool a fluid.

In other instances, the heat exchange element may be a fluid heat exchange element. By fluid heat exchange element is meant an element that is configured to position a fluid, e.g., liquid or gas, in heat exchange relationship with a target glabrous skin surface. In some instances, fluid heat exchange elements include a liquid flow path, i.e., a flow path configured for having a liquid, e.g., water, flow therethrough. In some embodiments, heat exchange elements include one or more e.g., one, or a plurality of, such as two, three, four, or more heat exchange domains, e.g., a flexible container having a high surface area, e.g., a polymeric container, and/or one or more inflow elements which may be operatively connected to the domain, and/or one or more outflow elements which may be operatively connected to the domain. By “operatively connected” and “operatively connect”, as used herein, is meant connected in a specific way, e.g., in a manner allowing fluid, e.g., water, to be transmitted therethrough and/or in a manner physically, e.g., adhesively, and/or electrically coupling, e.g., releasably or fixedly coupling, one aspect to another, that allows the disclosed devices to operate and/or methods to be carried out effectively in the manner described herein. As such, in some aspects, heat exchange elements are configured to receive fluid therein and/or therethrough and/or, in some embodiments, are configured to heat and/or cool a fluid.

As noted above, thermoregulatory devices and/or portions thereof, e.g., heat exchange elements, which may be employed with the methods, may include one or more heaters and/or coolers, e.g., an electrical heater and/or cooler. In some aspects, the heaters are configured, e.g., positioned, to heat one or more electrical elements, such as a coil and/or a resistor, and/or configured to heat a fluid, e.g., heating fluid, flowing through a thermoregulatory device or portions thereof, e.g., a container and/or inflow element and/or outflow element. In some aspects, the coolers are configured, e.g., positioned, to cool an electrical cooling component and/or a fluid, e.g., cooling fluid, flowing through a thermoregulatory device or portions thereof. In some embodiments, heat exchange elements and/or portions thereof, such as heaters, e.g., electrical heaters, and/or coolers are operatively connected, e.g., sealably connected, to a portion, e.g., an end, of an inflow element and/or an outflow element. In various embodiments, heaters and/or coolers have one or more cavities therein and/or are configured to receive a flow of fluid therethrough. In some embodiments, heat exchange elements and/or portions thereof, e.g., heaters and/or coolers, are operatively connected to one or more power sources, such as any of the power sources described herein, e.g., a battery. In various embodiments, heat exchange elements and/or portions thereof, such as heaters and/or coolers are connected within a circuit, e.g., a fluidic circuit, with other elements of thermoregulatory devices, e.g., containers and/or inflow elements and/or outflow elements and/or pumps. In those embodiments where the heat exchange element is a fluid heat exchange element, such as one that includes a liquid flow path, the methods may include flowing a liquid, e.g., water, having a desired temperature through the flow path in a manner sufficient to cause heat transfer the a target glabrous skin surface.

In some embodiments, the methods include turning one or more thermoregulatory devices or portion thereof, e.g., a heat exchange element and/or an electrical heater and/or cooler thereof, on and/or off, e.g., automatically, e.g., by using a controller, and/or manually by actuating an on/off switch. Certain aspects of the methods include controlling, e.g., manually and/or automatically controlling by increasing and/or decreasing and/or maintaining, the amount of electrical current passing through a heat exchange element or a portion thereof, such as one or more electrical element, such as a coil and/or a resistor. Additionally, in some aspects, the methods include adjusting the temperature, e.g., raising and/or lowering the temperature of one or more thermoregulatory devices or a portion thereof, e.g., a heat exchange element or a portion thereof, e.g., an electrical heater and/or cooler. Such an adjustment may be made automatically, e.g., by using a controller, and/or manually by actuating a switch. In addition, turning an a heat exchange element or a portion thereof, e.g., an electrical heater and/or cooler, on and/or off and/or adjusting the temperature of such a device may be performed, e.g., automatically and/or manually performed, based on an aspect such as a body core temperature measurement.

The methods, in various embodiments, include heating and/or cooling a fluid. On the other hand, the methods, in some embodiments, do not include heating and/or cooling a fluid. In some aspects, the methods include heating a fluid, e.g., a heating fluid, for example, by using a heater, e.g., an electrical heater. In some aspects, the methods include cooling a fluid, e.g., a cooling fluid, for example, by using a cooler, e.g., an electrical cooler. In some aspects, the methods include heating and/or cooling a fluid before then propelling the fluid into a container of a heat exchange element of a thermoregulatory device via, for example, an inflow element. In some variations, the subject methods include heating and/or cooling a fluid by 5° C. or less, 10° C. or less, or 25° C. or less. In certain embodiments, the temperature to which the fluids are heated and/or cooled is specifically selected to be one that provides for thermal energy introduction and/or extraction from a target location of a mammal, such as the circulatory system of the mammal.

Thermoregulatory devices that may be employed with the methods may also include one or more pumps, e.g., fluidic pumps, which may be electric pumps. In various embodiments, pumps are connected within a circuit, e.g., a fluidic circuit and/or an electrical circuit, with other elements of thermoregulatory devices, e.g., heat exchange elements, and/or containers, and/or inflow elements, and/or outflow elements, and/or heaters and/or coolers. In some aspects, pumps of thermoregulatory devices are configured to pump, e.g., propel, fluid, e.g., heating or cooling fluid, through, e.g., unidirectionally through, a circuit, e.g., a fluidic circuit, of the devices. In some embodiments, the pumps include an on/off switch, such as an automatic or manually actuable switch which is configured to turn the pump on and/or off. As such, the methods, in some aspects, include turning a pump on and/or off, e.g., electrically turning a pump on and/or off.

The methods, in some variations, include propelling, e.g., pumping, fluid, e.g., heating or cooling fluid, through a thermoregulatory device or one or more portions thereof, e.g., a heat exchange element, and/or a container, and/or an inflow element, and/or an outflow element, and/or a heater, and/or a cooler. In addition, some versions of the methods include initiating, and/or stopping, and/or maintaining propulsion of fluid through a thermoregulatory device or one or more portions thereof, e.g., heat exchange element, and/or a container, and/or an inflow element, and/or an outflow element, and/or a heater, and/or a cooler. In some instances, initiating and/or stopping propulsion of fluid through a thermoregulatory device or one or more portions thereof may include turning a pump and/or one or more valves, e.g., fluidic valves positioned within a circuit, e.g., a fluidic circuit of a thermoregulatory device, on and/or off.

In various aspects of the methods, the thermoregulatory devices employed include one or more attachment elements, such as any of the attachment elements described herein, e.g., an attachment element configured to secure a container to a heat exchange surface of a mammal. As such, in some versions of the methods, the attachment element of an employed device includes one or more, e.g., two, three, four, five, six, etc., or 10 or less, fasteners, e.g., a fastener having first and second joinable, e.g., removably and/or reversibly attachable, portions, such as a hook and loop fastener. The methods, in some variations, include attaching, e.g., reversibly affixing, a portion, e.g., a first portion, of an attachment element, e.g., a fastener, to a portion, e.g., a second portion, of an attachment element e.g., a fastener and/or a sheet. In other words, in some embodiments, the methods include attaching a first fastener, e.g., a hook and loop fastener, to a second fastener and/or a sheet of an attachment element. The subject methods, in some instances, include enveloping, e.g., wrapping, a portion, e.g., a hand and/or foot, of a limb, e.g., an arm and/or a leg, of a mammal with an attachment element or a portion thereof, e.g., a sheet. As such, the methods may include placing at least a portion of a mammal between two portions of an attachment element or portion thereof e.g., a sheet.

A thermoregulatory device or container thereof used in accordance with the subject methods may be configured, e.g., have one or more surfaces, e.g., body interfaces, for contacting and/or transferring heat with, e.g., only contacting, a portion of a mammal, such as a portion of a limb, e.g., an arm, and/or a wrist and/or a hand and/or a palm thereof, and/or a leg, and/or an ankle and/or a foot and/or a sole thereof, of a mammal. For example, such a device or container thereof may be configured, e.g., sized and/or shaped, to maintain physical contact with only a portion of a limb of a mammal, e.g., a heat exchange surface, instead of more than that particular portion of a mammal. As such, the subject methods, in various embodiments include contacting a thermoregulatory device with only a portion of a limb of a mammal, e.g., a hand and/or foot and/or one or more, e.g., two, three, four, etc., heat exchange surfaces. Accordingly, various aspects of the methods include limiting contact between a thermoregulatory device and a portion, e.g., limb, of a mammal to occur only at a portion of a limb of a mammal, e.g., a hand and/or foot and/or palm and/or sole. As such, the subject methods, in some embodiments, include not contacting and/or covering and/or wrapping around e.g., enveloping, a mammal's body or a portion, e.g., a substantial portion, thereof, e.g., a chest, with a thermoregulatory device or portion thereof. In some instances, only a target glabrous surface and its immediate portion of the limb, e.g., hand or foot, is contacted with the device, with remainder of the limb, e.g., arm, leg, not being contacted with the device. In various aspects of the methods, the thermoregulatory devices employed include one or more, e.g., two, three, four, etc., temperature monitors, e.g., a thermometer and/or an electronic temperature monitor. Such a temperature monitor may be configured to measure the temperature of a mammal, e.g., the body core temperature of a mammal. Temperature monitors may also be configured to measure the temperature of fluid within a thermoregulatory device, or portions thereof, e.g., heat exchange element, and/or a container and/or a heater and/or a cooler. As such, the subject methods may include monitoring, e.g., intra-operatively monitoring, the body core temperature of a mammal. The subject methods may also include monitoring, e.g., intra-operatively monitoring, the temperature of fluid within a thermoregulatory device or portion thereof, e.g., heat exchange element, and/or a container.

In various embodiments, the methods include monitoring and/or adjusting, e.g., elevating and/or lowering, the temperature of a thermoregulatory device or a portion thereof, e.g., heat exchange element, and/or a container or portion thereof, e.g., an outer surface of a container, and/or fluid flowing through a thermoregulatory device. Some embodiments of the methods include adjusting, e.g., manually and/or automatically adjusting, the temperature of a thermoregulatory device or a portion thereof, e.g., heat exchange element, and/or a container, based on the body core temperature of a mammal, e.g., a body core temperature recorded with a temperature monitor. For example, the methods may include heating the temperature of a thermoregulatory device or a portion thereof if a body core temperature of a mammal, e.g., a body core temperature recorded with a temperature monitor, is too low and/or thereby elevating the body core temperature of the mammal. In addition, the methods may include cooling the temperature of a thermoregulatory device or a portion thereof if a body core temperature of a mammal, e.g., a body core temperature recorded with a temperature monitor, is too high and/or thereby lowering the body core temperature of the mammal. In addition, the methods may include maintaining the temperature of a thermoregulatory device or a portion thereof if a body core temperature of a mammal, e.g., a body core temperature recorded with a temperature monitor, is within a desired range and/or thereby maintaining the body core temperature of the mammal within a temperature range, e.g., a range within 3° C., 5° C., or 10° C., of a desired temperature.

In addition, various thermoregulatory devices employed with the subject methods may include one or more control units, e.g., a control unit having the characteristics or features of any control unit described herein, configured to adjust, e.g., automatically adjust, the temperature of a thermoregulatory device or a portion thereof, e.g., heat exchange element, and/or a container, based on the body core temperature of a mammal.

Various embodiments of the methods may include adjusting, e.g., manually and/or automatically adjusting, the temperature of a device, e.g., an electrical device, or a portion thereof, e.g., a heat exchange element, and/or fluid, e.g., fluid flowing through a thermoregulatory device or a portion thereof, e.g., a container, based on the body core temperature of a mammal, e.g., a body core temperature recorded with a temperature monitor. For example, the methods may include increasing the temperature of a heat exchange element and/or fluid if a body core temperature of a mammal, e.g., a body core temperature recorded with a temperature monitor, is too low and/or thereby elevating the body core temperature of the mammal. In addition, the methods may include cooling the temperature of heat exchange element and/or fluid if a body core temperature of a mammal, e.g., a body core temperature recorded with a temperature monitor, is too high and/or thereby lowering the body core temperature of the mammal. In addition, the methods may include maintaining the temperature of a heat exchange element and/or fluid if a body core temperature of a mammal, e.g., a body core temperature recorded with a temperature monitor, is within a desired range and/or thereby maintaining the body core temperature of the mammal within a temperature range, e.g., a range within 3° C., 5° C., or 10° C., of a desired temperature.

In various embodiments of the methods, a portion of the mammal, e.g., a portion of a mammal with which heat is transferred, e.g., a limb and/or hand and/or foot and/or palm and/or sole, is not under negative pressure. As used herein, the phrase “negative pressure” refers to a pressure lower than ambient pressure under the particular conditions in which the device or system is employed or the method is performed, e.g., 760 mmHg at sea level. In some embodiments of the methods, a negative pressure element and/or a device or system having such an element is not employed. The phrase “negative pressure element”, as used herein, refers to a device and/or system element configured for inducing and/or maintaining negative pressure, e.g., negative pressure within a specific enclosed area of the device, such as an area enclosed by a portion of a negative pressure element. Such a negative pressure element may be configured for applying negative pressure for a length of time. Accordingly, in some embodiments, the subject devices, systems and methods do not include creating a vacuum, e.g., a state of negative pressure, around or proximate a heat exchange surface of a mammal when, for example, heat is transferred through the surface.

In some instances, the mammal has a compromised thermoregulatory system. As used herein, the phrase “compromised thermoregulatory system” refers to a thermoregulatory system of a mammal which has been modified, e.g., temporarily and/or reversibly modified, from its initial, e.g., normally functioning natural state, to prevent and/or substantially prevent local vasodilation and/or vasoconstriction, e.g., vasoconstriction at a heat exchange surface of a mammal, such as, for example, vasoconstriction occurring upon exposure of a portion of a mammal to a medium having a lower temperature, e.g., a substantially lower temperature, than that portion of the mammal. Such mammals may also be referred to as mammals having compromised vasoconstriction.

In some embodiments, the methods include compromising the vasoconstriction in the mammal, e.g., at least in the target glabrous surface thereof. Vasoconstriction may be compromised using any convenient approach. For example, a thermoregulatory system/vasoconstriction of a mammal may be compromised by producing anesthesia in the mammal or a portion of thereof, e.g., the portion having the target glabrous surface. Anesthesia may be produced using any convenient protocol, e.g., by administering an anesthetic agent, e.g., a general and/or local anesthetic agent, to a mammal. Accordingly, in various embodiments of the subject methods, the subject mammal is a mammal under anesthesia. Examples of general anesthetic agents which may be administered to a mammal to compromise its thermoregulatory system include, but are not limited to: propofol, sodium thiopental, etomidate, ketamine and sevoflurane.

In various embodiments of the methods, the subject mammal is a mammal under anesthesia and the methods include initiating and/or maintaining, and/or terminating anesthesia in a mammal. Accordingly, various aspects of the methods include initiating and/or maintaining and/or terminating the administration of an anesthetic agent, e.g., a general and/or local anesthetic agent, to a mammal and/or compromising the thermoregulatory system of a mammal.

In some embodiments, the methods include decreasing the health risk posed by a surgical procedure by, for example, maintaining a core body temperature of a mammal within a desired range. Certain embodiments of the methods include treating a mammal by applying the subject devices and/or systems to cause a mammal, e.g., a mammal undergoing a surgical procedure, to have a reduced healing time and/or a faster recovery from a surgical procedure than the mammal would without such treatment.

In various embodiments, the methods include increasing and/or decreasing and/or maintaining, e.g., intra-operatively increasing and/or decreasing and/or maintaining, the core body temperature of a mammal. In practicing the subject methods, heat is introduced to and/or extracted from a portion of a mammal, e.g., the body core of a mammal, at least once during a surgical procedure to result in the desired elevation and/or reduction and/or maintenance of the mammal's body core temperature. The magnitude of core body thermal energy introduced or extracted may vary, and is sufficient to provide for the desired outcome, e.g. a safer surgical procedure, a reduced healing time, less bleeding, etc., and the like. In certain embodiments, the magnitude of heat introduction and/or extraction is 0.5 Kcal/min or less, 1.0 Kcal/min or less, or 1.5 Kcal/min or less, where the magnitude may be 50 Kcal/min or greater, but sometimes is 30 Kcal/min or less, such as 20 Kcal/min or less. The period of time that the heat is introduced and/or extracted may range, for example, from 1 min to 24 hrs, such as from 2 min to 20 min, or from 2 min to 5 min.

As noted below, in certain embodiments, the core body temperature of a mammal is elevated and/or reduced and/or maintained. The magnitude of core body temperature elevation and/or reduction is sufficient to provide for a desired outcome, e.g., a safer surgical procedure, and is, in some aspects, 0.5° C. or less, or 1.0° C. or greater, or 1.5° C. or greater, or may be 4° C. or greater, or 4.0° C. or less, or 2.0° C. or less. The period of time that the core body temperature is elevated and/or reduced and/or maintained may range from 1 min to continuous for the duration of a surgical procedure. For example, the period of time that the core body temperature is elevated and/or reduced and/or maintained may vary, ranging in some instances from 2 min to 720 min, such as 15 to 600 minutes, e.g., 60 to 300 minutes. In some embodiments, the subject methods will prevent or minimize rises or drops in the core body temperature, e.g., where any drop may be 2° C. or less, such as 1.5° C. or less, including 1° C. or less, e.g., 0.75° C. or less, such as 0.5° C. or less.

In some instances, the methods are intra-operative methods. In addition, the phrase “intra-operatively” refers to taking place during a surgical procedure, e.g., a surgical procedure within an operating room. As such, the subject methods include transferring heat with, e.g., transferring heat to and/or from, a portion of a mammal, e.g., a heat exchange surface, during a surgical procedure. Such a surgical procedure can include the actual procedure itself, as well as the period of time during preparation for and after the conclusion of the surgical procedure. In addition, the subject methods, in various aspects, include performing one or more surgical procedures on a mammal.

In certain embodiments, core body heat is intra-operatively transferred, e.g., transferred through a heat exchange surface of a portion of a mammal, one or more, e.g., a plurality, of times. Where heat is transferred a plurality of times, the number of different times that heat is introduced and/or extracted may, for example, range from 2 to 500, such as from 2 to 100, such as from 5 to 50. In certain embodiments, heat is transferred a single time.

In certain embodiments, the devices employed in the subject methods further include a feedback element that at least partially controls a temperature of a thermoregulatory device and/or portion thereof, e.g., a container, and/or when a heat exchange surface of a mammal is contacted and/or stably associated with a thermoregulatory device to transfer heat with a portion of a mammal and/or to introduce and/or extract thermal energy from the core body of the mammal. The feedback element may be any convenient element, where a suitable element is a temperature monitor, e.g., a thermosensor, e.g., placed over a heat exchange surface not being contacted with thermoregulatory device. In such embodiments, the method, in some aspects, further includes a data processing step for processing the feedback data and activating the temperature control and/or contact and/or stable association with a thermoregulatory device or portion thereof in response thereto, e.g. a computing element that controls the contact of the heat exchange surface with the low temperature medium. Additionally, as noted above, the subject methods are suitable for use with a variety of mammals.

A given method may include transferring heat through only one glabrous surface of a mammal or through two or more distinct glabrous surfaces, e.g., three or more, including four or more distinct glabrous surfaces of the mammal. For example, a given method may include stably associating a heat exchange device with each palm of the mammal and/or each sole of the mammal. In some instances, a heat exchange device is associated with each palm and each sole of the mammal, such that the mammal has four distinct heat exchange devices associated with it.

The methods described here find use with a variety of different mammals. The terms “mammal” and “mammals” are used broadly herein to describe organisms which are within the class mammalia, including the orders carnivore (e.g., dogs and cats), rodentia (e.g., mice, guinea pigs, and rats), and primates (e.g., humans, chimpanzees, and monkeys). Mammals may be subjects or patients, such as human subjects or patients. The terms “human” or “humans” may include human subjects or patients of both genders and at any stage of development (i.e., fetal, neonates, infant, juvenile, adolescent, adult), where in certain embodiments the human subject is a juvenile, adolescent or adult. While the systems, devices and methods described herein may be applied on a human subject, it is to be understood that the subject systems, devices and methods may also be applied on other subjects (that is, in “non-human subjects”).

### Devices

Aspects of the invention include thermoregulatory devices configured for use in methods of transcutaneous heat transfer, e.g., as described herein. As used herein, the terms “heat exchange device”, “thermoregulatory device” and “thermoregulatory devices” refer to one or a plurality of devices which are configured to be used to transfer heat across a glabrous surface of a mammal. The devices may be configured to transfer heat into or out of a internal region, such as a body core, of a mammal. By “body core” and “core body”, as used herein, is meant the internal region of a mammal, as opposed to the surface of a mammal.

Thermoregulatory devices of interest include one or more heat exhange elements and one or more attachment elements, which elements are described in greater detail below. In some versions, the thermoregulatory devices include one or more, e.g., two, three, four, etc., heat exchange elements. As described in greater detail below, in various instances, heat exchange elements are configured for transferring heat through a heat exchange, e.g., glabrous, surface of a portion of a mammal. As described above, the phrase “heat exchange surface” refers to a surface of a mammal through which heat may transfer, e.g., readily transfer. Heat exchange surfaces which are of interest with the subject systems, devices and methods include those found in various regions of the mammal, e.g., extremities and/or head. For example, such portions may include one or more of the arms, legs, palms, soles, and the like, or any combination thereof. As such, heat exchange surfaces may be the palms and/or soles of a mammal.

Heat exchange elements of the subject devices may be heating and/or cooling elements. Heating elements, e.g., elements including one or more heater, are elements that are configured to introduce heat to a portion, e.g., a body core, of a mammal whereas cooling elements, e.g., elements including one or more cooler, are elements that are configured to remove heat from a portion of a mammal. As such, a heating element is configured to cause energy transfer into a mammal from the heating element upon contact (e.g., physical contact and/or thermal exposure) of the heating element with a heat exchange surface of the mammal. Embodiments of subject cooling elements are configured to contact a portion of a mammal, such as a heat exchange surface and operate to remove heat from the mammal, for example, to lower the core body temperature of a mammal. As such, a cooling element is configured to cause energy transfer from a mammal, to the cooling element upon contact of the cooling element with a heat exchange surface of the mammal.

As noted above, in various embodiments, heat exchange elements include one or more heaters and/or coolers, e.g., an electrical heater and/or cooler, such as a heater and/or cooler having one or more electrical components, e.g., an electrode and/or a resister and/or a coil. Heat exchange elements, in various aspects, are not configured to receive fluid therein and/or therethrough and/or, in some embodiments, are not configured to heat and/or cool a fluid.

In some embodiments, heat exchange elements include one or more e.g., one, or a plurality of, such as two, three, four, or more fluid, e.g., liquid flow paths, which flow paths may include a high surface area heat transfer domain, such as may be provided by a suitable dimensioned liquid container, e.g., a flexible container, e.g., a polymeric container, and/or one or more inflow elements which may be operatively connected to the container, and/or one or more outflow elements which may be operatively connected to the container. As such, in some aspects, heat exchange elements are configured to receive fluid therein and/or therethrough and/or, in some embodiments, are configured to heat and/or cool a fluid.

In various embodiments, the subject thermoregulatory devices are devices for transferring heat with a portion of a mammal, e.g., a limb and/or hand and/or foot and/or palm and/or sole, and the portion of the mammal is not under negative pressure, e.g., held under negative pressure conditions, when, for example, heat is transferred. As noted above, the phrase “negative pressure” refers to a pressure lower than ambient pressure under the particular conditions in which the device or system is employed or the method is performed, e.g., 760 mmHg at sea level. As such, in some versions, the portion of a mammal with which heat is transferred is under atmospheric and/or ambient pressure. In some versions, the subject devices and/or systems do not include one or more elements, e.g., a container, configured to maintain a portion of a mammal, e.g., a portion of a mammal with which heat is transferred, such as a palm and/or sole, under negative pressure conditions.

In various embodiments, the thermoregulatory devices are configured, e.g., sized and/or shaped, for surgical operating room use. As such, in certain variations, the devices may be utilized in or out of the sterile field, but in certain instances the devices are utilized in a room where a given surgical procedure is performed. As such, in some aspects, the devices are configured for intra-operative use. As used herein, the phrases “intra-operatively” and “intra-operative”, refer to taking place during a surgical procedure. Accordingly, by “intra-operative use”, as used herein, is meant use during a surgical procedure, e.g., a surgical procedure within an operating room. As such, a device may be configured to transfer heat, e.g., transfer heat to and/or from, with a heat exchange surface of a mammal during a surgical procedure on the mammal. By “surgical procedure” is meant an operation, including an open surgical procedure, or a minimally-invasive surgical procedure, including endoscopic procedures, such that there is at least one opening into a body, e.g., a body of a mammal. A surgical procedure can include the actual procedure itself, as well as the period of time during preparation for and after the conclusion of the surgical procedure.

In various instances, the subject thermoregulatory devices are thermoelectric devices, e.g., devices having one or more electrical components and powered by an electrical power source. Such electrical devices or components thereof, e.g., heat exchange elements, may include, for example, electrical heaters and/or electrical coolers. The electrical heaters and/or coolers may in turn may be integrated into, e.g., contained fully or partially within, at least a portion of a device, such as a container and/or another portion described herein. Additionally, the electrical heaters may include one or more electrical heating components, e.g., an electrical heating coil, such as a metallic coil. Furthermore, the electrical coolers may include one or more electrical cooling components, e.g., Peltier devices. Electrical heaters and/or coolers may be secured inside another portion of a device, e.g., a container, by a holder, e.g., a holding pad.

In some embodiments, heat exchange elements, or portions thereof, such as electrical heaters and/or coolers, are operatively connected (e.g., electrically coupled) to a power source. A power source may, in some aspects, be a battery, e.g., a portable and/or self-contained battery, an outlet, or another source of electrical power. In some aspects, heat exchange elements, or portions thereof, such as electrical heaters and/or coolers may include one or more electrical cords, e.g., cords configured to operatively connect a device to a power source, such as an outlet. Heat exchange elements, or portions thereof, e.g., electrical heaters and/or coolers, in various versions, also include an on/off switch configured, for example, to turn the electrical heater and/or cooler on and/or off. Such a switch may be manual and/or automatic. Heat exchange elements, or portions thereof, e.g., electrical heaters and/or coolers also may include a switch, e.g., a manual and/or automatic switch, to control the temperature of the element. In some aspects, heat exchange elements, or portions thereof, such as electrical heaters and/or coolers are operatively connected to a controller, such as any of the controllers described herein, which may be configured to adjust, e.g., automatically adjust, e.g., adjust based on a core body temperature measurement, the temperature of the heater and/or coolers.

In various embodiments, the temperature at which heat exchange elements, or portions thereof, such as electrical heaters and/or coolers may be maintained, e.g., maintained for a period of time during operation, may range, for example, from 1° C. to 50° C., such as from 15° C. to 45° C., including from 30° C. to 40° C. In addition, electrical heaters may be heated to a temperature higher, e.g., 5° C. or more; 10° C. or more; or 20° C. or more, than the core body temperature of a mammal, e.g., 37° C., and thereby may provide for the introduction of heat into the body of a mammal, for example, through a heat exchange surface thereof. Furthermore, electrical coolers may be cooled to a temperature lower, e.g., lower by 5° C. or more; 10° C. or more; or 20° C. or more, than the core body temperature of a mammal, and thereby may provide for the extraction of heat from the body of a mammal, for example, through a heat exchange surface thereof. Additionally, heat exchange elements, or portions thereof, such as electrical heaters and/or coolers may include one or more of the characteristics of the other devices described herein, e.g., non-electrical devices, or any combination of such characteristics and/or may be used according to any combination of the methods described herein.

As noted above, in certain embodiments, the subject thermoregulatory devices, or portions thereof, such as heat exchange elements, include one or more e.g., one, or a plurality of, such as two, three, four, or more, containers. A thermoregulatory device, heat exchange element, or container thereof may be configured, e.g., have one or more surfaces, e.g., body interfaces, for contacting and/or transferring heat with, e.g., only contacting, a portion of a mammal, such as a portion of a limb, e.g., an arm, and/or a wrist and/or a hand and/or a palm thereof, and/or a leg, and/or an ankle and/or a foot and/or a sole thereof, of a mammal. For example, such a device, element, or container thereof may be configured, e.g., sized and/or shaped, to maintain physical contact with only a portion of a limb of a mammal, e.g., a heat exchange surface, instead of more than that particular portion of a mammal. In addition, a thermoregulatory device, heat exchange element, or container thereof may be configured for contacting, e.g., only contacting, a heat exchange surface, e.g., a palm and/or sole, of a mammal, such as any of the heat exchange surfaces described herein. As such, the subject thermoregulatory devices, heat exchange elements, or containers thereof, in some embodiments, do not contact and/or wrap around e.g., envelope, a mammal's body or a portion, e.g., a substantial portion, thereof, e.g., a chest. Additionally, in some embodiments, the containers are or include one or more water perfusion pads.

In addition, thermoregulatory devices, heat exchange elements, and the containers thereof may have any convenient size or shape to operate for the purposes described herein. As such, a container of a device may be formed as a substantially planar sheet, e.g., a hollow sheet having a plurality of layers with at least one cavity therebetween, such as a sheet configured to be wrapped around a portion of a mammal. A container of a device may also be shaped as a pouch having an opening for receiving a portion of a mammal therein. In certain aspects, containers are shaped substantially like a glove, a mitten, or a sock. In some aspects, containers have a continuous exterior wall enveloping an interior cavity. Various embodiments of containers have an outer periphery defining a shape which is, or substantially is, that of a circle, oval, rectangle (e.g., a rectangle having rounded corners), square, triangle, or any combination thereof. Certain variations of containers have an outer periphery defining a single continuous edge. In some embodiments, containers include a continuous inner surface, e.g., an inner surface having no distinguishable edges, and/or a single interior edge, e.g., a curving edge defining a periphery of the interior surface of the container.

The volume of the container of a device may vary, ranging in some instances from 5 cm³ to 40 cm³, such as from 10 cm³ to 30 cm³, including from 10 cm³ to 20 cm³. Such a volume may result in a container being sized to fit against, e.g., to contact, 95% of human hand sizes and/or 95% of human foot sizes. Alternately, a container may be sized for more specific groups, such as children.

In some embodiments, the containers have a length, width and height. The length and/or width of a container may be longer e.g., significantly longer, than the height of a container. In some embodiments, the length is a distance from a first edge, e.g., peripheral edge, of a container to a second edge of the container that is opposite the first edge. In certain embodiments, the width of a container is a distance from a third edge of a container to a fourth edge of the container that is opposite the third edge and may be measured in a direction transverse to the length of the container. Likewise, the height of a container may be the vertical height and/or a distance from a fifth edge of a container to a sixth edge of the container that is opposite the fifth edge and may be measured in a direction transverse to the length and width of the container. In some aspects, the length of a container is longer than the width of a container. In some aspects, the length of a container is equal to the width of a container. In some aspects, the length of a container may range from 5 cm to 40 cm, such as from 10 cm to 30 cm, such as from 15 cm to 25 cm. In addition, the width of a container may range from 5 cm to 40 cm, such as from 10 cm to 30 cm, such as from 15 cm to 25 cm. Furthermore, the height of a container may range from 0.5 cm to 20 cm, such as from 1 cm to 10 cm, such as from 1 cm to 5 cm.

Embodiments of the subject containers include a cavity therein. As such, in some aspects, the subject containers are hollow and/or are configured to receive a flow of fluid therethrough. Accordingly, in some embodiments, the subject containers include one or more containers, e.g., a pad, through which fluid, e.g., water, may flow. A container may include one compartment or a plurality, e.g., two, three, four, five, ten or fewer, fifty or fewer, one-hundred or fewer, such as between five and one-hundred, such as between forty and sixty, such as between forty-five and fifty-five, of compartments therein. Such compartments may each be connected by one or more openings through which fluid may flow. Accordingly, in some embodiments, the cavity of container is configured to allow to flow into the cavity through a first opening, e.g., through the inflow element, and out of the cavity through a second opening, e.g., through the outflow element. In some embodiments, the containers are air-tight and/or water tight but for an opening or openings where the container is operatively connected to the inflow and/or outflow elements.

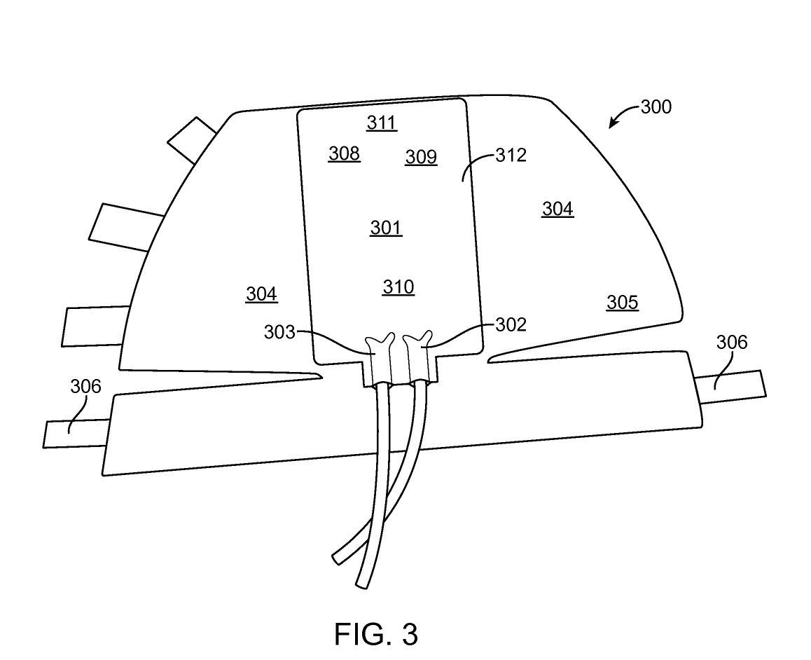

In some embodiments, cavities of containers include a first portion, e.g., a first compartment, operatively connected to an inflow element and a second portion e.g., a second compartment, operatively connected to an outflow element. The first and second portions may be separated by a wall of the container, e.g., a wall providing a seal to fluid, but for one or more, e.g., two, three, four, etc., openings between the first and second portions, e.g., an opening configured so that fluid can flow between the first to the second portions. For example, FIG. 3 provides an exterior view one embodiment of a container 301 having therein a first portion 308 of a container cavity, which is labelled for illustrative purposes in FIG. 3 on the exterior of the device which encloses the cavity, as well as a second portion of a container cavity 309, a wall 310 and an opening 311 between the first and second portion. In some embodiments, the inflow and outflow elements are located on a first end of a container and/or one or more openings between first and second portions of a container are located on a second end of a container which is opposite from the first end. As such, in some embodiments, the containers are configured such that fluid circulates through the container. For example, fluid may flow into a container through the inflow element at a first end of the container, and/or through a first portion of a container e.g., toward the second end of the container, and/or through an opening at a second end of the container to a second portion of a container and/or through a second portion of a container, e.g., toward the first end of the container, and/or out of the container through the outflow element.

In certain embodiments, containers are flexible containers. For instance, a flexible container may be compliant, for example, such that it is configured to form a form-fitted surface which is complementary with a portion of a mammal, such as a heat exchange surface, against which it is placed. In various embodiments, containers, such as flexible containers, are polymeric containers. Polymeric containers are containers which have one or more portions composed of one or more polymeric materials. Polymeric containers may be entirely or partially composed of one or more polymeric materials, e.g., a single polymeric material. Specific polymeric materials of interest include, but are not limited to: plastics, rubbers, silicones, etc.

In various embodiments, containers are rigid containers. For instance, a rigid container may be non-compliant, or substantially non-compliant, for example, such that it does not change its shape when contacted with an aspect, such as a portion of a mammal, such as a heat exchange surface, against which it is placed. In various embodiments, containers, such as rigid containers, are polymeric containers.

Certain aspects of thermoregulatory devices or portions thereof, e.g., heat exchange elements and/or containers and/or inflow elements, and/or outflow elements, and/or attachment elements, are disposable. In other words, the devices or portions thereof are configured for disposal following one or more uses, such as a single use. Embodiments of devices and the components thereof, e.g., a container and/or an attachment element, are composed of one or more biodegradable, e.g., organic, materials.

As noted above, in various embodiments, the subject thermoregulatory devices include a container and inflow and outflow elements operatively connected thereto. In various aspects, the subject devices include one or more (e.g., two, three, four, five, ten, twenty, or more than twenty) inflow elements and/or one or more (e.g., two, three, four, five, ten, twenty, or more than twenty) outflow elements. In some embodiments, the inflow and/or outflow elements of a device extend inside the container. In some embodiments, the container of a device extends inside the inflow and/or outflow elements. In various embodiments, the inflow and/or outflow elements are rigid or flexible and composed of any of the same materials as a container of a device.

In some embodiments of the devices, the inflow and/or outflow elements are integrated with the container. In such embodiments, because the container and inflow and/or outflow elements are integrated components, they cannot be separated from each other without altering the physical structure of one or both of the elements. For example, a container and an inflow element and/or outflow element may be composed of a single piece of continuous material. For example, a container may be fixedly attached to the inflow element and/or outflow element in a manner such that the container cannot be removed from the inflow element and/or outflow element without in some way altering the physical structure of the container. In embodiments wherein a container and inflow and/or outflow elements are integrated, they are not configured to operate separately from each other for their intended purpose, e.g., to contact a surface of a mammal with a surface, e.g., a reduced temperature surface, e.g., as described in greater detail below.

In some embodiments of the devices, the inflow and/or outflow elements are removably attached with the container. In such embodiments, the inflow and/or outflow elements are configured so that they may be repeatedly detached from the container and re-attached to the container.

In certain aspects of the subject devices, or portions thereof, such as heat exchange elements, or portions thereof, e.g., the container, inflow element and/or outflow element, are configured for receiving a flow of fluid, e.g., a heating or cooling fluid, therethrough. As such, in certain embodiments, the inflow and/or outflow elements are sealably attached with the container. For example, in some embodiments, the inflow and/or outflow elements are attached with the container so that fluid can flow between the inflow and/or outflow elements and the container without escaping the device. Additionally, in various embodiments, the inflow and/or outflow elements are adhesively and/or snappedly connected with a container.

Various embodiments of the subject devices include one or more inflow elements at a first end of a container and one or more outflow elements at the first end of the container and/or at a second end of the container, which is opposite the first end.

In some aspects the inflow and/or outflow elements, together with a container, form an interior cavity or cavities through which fluid may flow. Such a cavity or cavities may be continuous, e.g., in fluidic communication, with one or more cavities of a container, as described above.

The subject devices also, in some embodiments, include one or more (e.g., two, three, four, five or more, etc.) fluid conveying elements. Such fluid conveying elements are configured for receiving a flow of fluid, e.g., a heating or cooling fluid, therethrough. In some aspects, fluid conveying elements provide a cavity which is sealed between a first opening at a first end and a second opening at a second end. In some embodiments, fluid conveying elements are tubes.

In certain embodiments, the devices include the same number of fluid conveying elements as inflow and outflow elements. As such, in various embodiments the inflow and/or outflow elements are each connected to, e.g., operatively connected to, e.g., sealably and/or adhesively connected, e.g., in fluidic communication with, one or more fluid conveying elements. In various embodiments, a fluid conveying element connected to the inflow element at a first end of the element is operatively connected e.g., sealably and/or adhesively connected, at a second end to a source of heating fluid. In some embodiments of the devices, a fluid conveying element connected to the outflow element at a first end of the element is operatively connected, at a second end to a waste container and/or the source for producing heating or cooling fluid. As such, in some embodiments, the devices are configured so that fluid can be re-circulated through the devices by causing the fluid to flow out of the container through an outflow element, heating or cooling the fluid, and subsequently causing the fluid to flow into the container through an inflow element.

In some embodiments, the one or more fluid conveying elements, e.g., tubes, are integrated with the inflow and/or outflow elements and/or the container of a device. In some embodiments, the fluid conveying elements are flexible tubes. In various embodiments, the fluid conveying elements are composed of any of the same materials as the inflow element and/or the outflow element, and/or the container of a device.

Various embodiments of the subject devices include fluid conveying elements, e.g., tubes, which are configured, e.g., sized and/or shaped to perform according to any of the subject methods. In some embodiments, the fluid conveying elements, e.g., tubes, extend longitudinally from a first end to a second end and/or have circular cross-section along the length of each element.

As noted above, in certain aspects of the subject devices, the container and/or inflow element and/or outflow element and/or one or more fluid conveying elements, e.g., tubes, are configured for receiving a flow of fluid, e.g., a heating or cooling fluid, therethrough. Such a fluid may be any suitable fluid having the characteristics, e.g., viscosity, to operate within the devices and systems disclosed herein. In certain embodiments of heating and/or cooling fluids, the fluids are composed entirely of or include water.

In the broadest sense, the subject fluid, e.g., the heating and/or cooling fluid, may be a composition that is capable of attaining a temperature that, upon contact of the device or portion thereof, e.g., container, with the heat exchange surface of a mammal, results in transfer of energy from the mammal to the fluid or from the fluid to the mammal. The fluid may be a gas or liquid, and may or may not include water.

In some embodiments, the fluid which the portions of the disclosed devices are configured to receive is a fluid, which may be heated to a temperature higher, e.g., 5° C. or more; 10° C. or more; or 20° C. or more, than the core body temperature of a mammal, e.g., 37° C. Such a fluid is referred to herein as a heating fluid and may provide for the introduction of heat into the body of a mammal, for example, through a heat exchange surface thereof.

In certain variations, the fluid which the portions of the disclosed devices are configured to receive is a fluid which may be cooled to a temperature lower, e.g., lower by 5° C. or more; 10° C. or more; or 20° C. or more, than the core body temperature of a mammal. Such a fluid is referred to herein as a cooling fluid and may provide for the removal of heat from the body of a mammal, for example, through a heat exchange surface thereof. In some embodiments, a fluid may operate as a heating fluid at a first time during use of a device and operate as a cooling fluid at a second time during use of a device.

In various embodiments, the temperature at which a subject fluid is configured to be maintained, e.g., heated and/or cooled to, may range, for example, from 1° C. to 50° C., such as from 10° C. to 45° C., including from 25° C. to 42° C. The subject fluids may be exposed, e.g., thermally exposed, to a portion of a mammal, e.g., a portion of a limb of a mammal, for a period of time, e.g., a period of time necessary that the mammal is under anesthesia and/or a period of time necessary to complete a surgical procedure on the mammal. Such contact or exposure may be through a portion of a container containing the fluid which is positioned immediately adjacent to or contacting a portion of a mammal. A period of time of exposure or contact between the fluid and a portion of a mammal may be a period of time required for energy, such as thermal energy, such as thermal energy from a portion of a mammal, to be absorbed by the fluid. A period of time of exposure or contact between the fluid and a portion of a mammal may be a period of time required for energy, such as thermal energy, such as thermal energy from the fluid, to be absorbed by the mammal. Additionally, the subject fluids may assume the shape of the interior of the container of the device in which the fluid is contained. Certain variations of the subject fluids are configured to maintain the environment immediately surrounding (e.g., contacting, and/or within 1 cm, 2 cm, 5 cm, 10 cm, or 20 cm, or within a container) the fluid at the same temperature, or substantially the same temperature (e.g., within 1° C., 2° C., or 5° C.), as that of the fluid. As noted above, the temperature of the subject fluids, e.g., fluids within a container of a device may vary. In certain embodiments, fluids have a temperature, which is a temperature that provides for thermal energy extraction from, or thermal energy introduction to, the core body of a mammal and not local vasoconstriction and/or vasodilation.

In various aspects, the subject devices are configured such that exposure, e.g., thermal exposure, may be maintained between the fluids and a mammal for a period of time sufficient for a desired amount of core body thermal energy extraction or introduction to occur. Such a period of time may be, for example, 1 min or less, 2 min or less, 3 min or less, or 5 min or less, and/or such contact may be maintained for 10 hour or less, such as 1 hour or less, such as 5 min or less, e.g., for the duration of a surgical procedure or less.

Embodiments of subject thermoregulatory devices include one or more, e.g., one, such as only a single one, or a plurality, such as two, three, four, or more, attachment elements. Such an attachment elements may be configured to secure a heat exchange element of a device to a portion of a mammal, e.g., a glabrous surface, in a heat transfer relationship. In securing a container to a portion of a mammal, the attachment element may cause the container to be retained in a position immediately adjacent to and/or contacting and/or pressing against, the portion of the mammal for the duration of use of the device, e.g., for the duration of a surgery on the mammal, or longer.

In various embodiments, an attachment element, or portion thereof, e.g., a sheet and/or a fastener and/or an attaching band, is configured to secure a heat exchange element or a portion thereof, e.g., a container to a portion of a mammal, e.g., a hand or foot. In some aspects, attachment elements include one sheet or a plurality, e.g., two, three, four, etc., of sheets. As such, in some embodiments, attachment elements are operatively connected to heat exchange elements of a device.

In some embodiments a sheet of an attachment element has a thin and/or planar shape. In various embodiments, a sheet is or includes a uniform layer of a single material, e.g., a fabric. A sheet may, in some aspects, have a thickness of 1 cm or less or 0.5 cm or less or 0.3 cm or less. A sheet may be flexible and/or may be configured to wrap fully or partially around, e.g., envelope, a portion of a mammal, e.g., a hand and/or foot. In some embodiments, the volume that an attachment element of a device is configured to wrap around, envelop, or contain, may vary, ranging in some instances from 5 cm³ to 40 cm³, such as from 10 cm³ to 30 cm³, including from 10 cm³ to 20 cm³. Such a volume may be sized to fit against, e.g., to contact, 95% of human hand sizes and/or 95% of human foot sizes. Alternately, it may be sized for more specific groups, such as children.

As noted above, attachment elements or portions thereof, e.g., sheets, may be configured to produce, or hold and/or receive, an enclosed portion of a mammal, e.g., only a portion of a limb of a mammal. As used herein, the phrase “enclosed portion of a mammal” refers to a portion of a mammal, e.g., a wrist and/or hand, which lies within, or substantially within, such as between two or more portions, e.g., opposite interior portions of, or encapsulated within, an attachment element. An enclosed portion of a mammal may be a portion of a mammal that is contained within or surrounded by an attachment element. An enclosed portion of a mammal may be, for example, one or more of a hand, and/or a foot, and/or an arm, and/or a leg, and/or a finger, and/or a head, and the like, or any combination thereof. An enclosed portion of a mammal may include one or more heat exchange surfaces of a mammal.

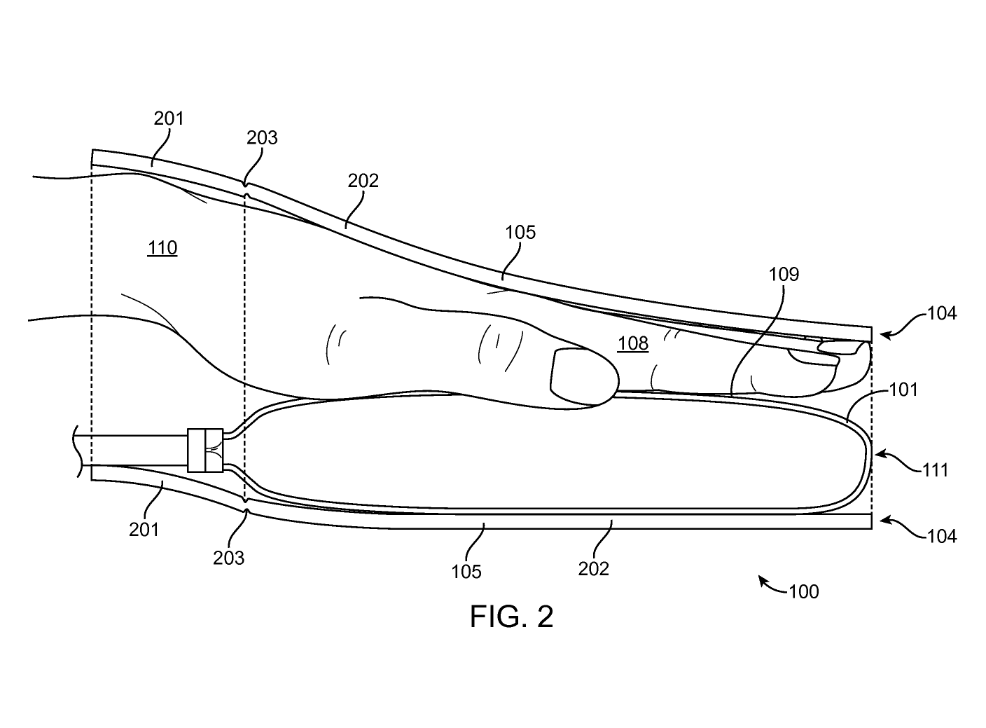

In some embodiments, a sheet of an attachment element has a first portion, e.g., a portion configured to wrap around a first portion of a mammal, such as a wrist and/or ankle, and a second portion, e.g., a portion configured to wrap around a second portion of a mammal, such as a hand and/or foot. For example, one embodiment of a first portion of an attachment element is shown in FIG. 2 as element 201 and one embodiment of a second portion of an attachment element is shown in FIG. 2 as element 202. In some aspects, the first portion of the sheet is at a first end of the sheet and the second portion of the sheet is at a second end of the sheet which is opposite the first end. In some aspects, the first portion of the sheet is configured to wrap around, e.g., envelop a container of a device and the second portion of the sheet is configured to wrap around, e.g., envelop, the inflow and/or outflow elements and/or one or more fluid conveying elements, e.g., tubes, e.g., two tubes, operatively connected thereto.

In some embodiments, a portion e.g., a second portion of a sheet, is tapered toward, e.g., has a width that narrows toward the second end. In various instances, the sheets have a length extending, for example, from the first end to the second end of a sheet and width extending transversely to the length. In some embodiments, the length is longer than the width. In some aspects, the first end of a sheet is separated from the second end of the sheet by one or more, e.g., two, e.g., two opposite, cuts extending, e.g., extending in a direction transverse to the length of the device, at least partially across the sheet. For example, one embodiment of a cut between first and second portions of an attachment element is shown in FIG. 2. as element 203. In some embodiments, the cuts have the same size and shape. In addition, in certain configurations, sheets are symmetrical about one and/or two and/or three planes, e.g., planes extending through the center of the sheet.

Various embodiments of sheets and/or portions thereof, e.g., a first and/or second portion, have an outer periphery defining a shape which is, or substantially is, that of a circle, oval, rectangle (e.g., a rectangle having rounded corners), square, triangle, or any combination thereof. Certain variations of sheets have an outer periphery defining a single continuous edge.

In some embodiments, the attachment elements and the portions thereof, e.g., sheets, have a length, width and height. The length and/or width of an attachment elements and the portions thereof, e.g., a sheet, may be longer e.g., significantly longer, than the height of an attachment elements or portion thereof. In some embodiments, the length is a distance from a first edge, e.g., peripheral edge, of an attachment elements or portion thereof to a second edge of the attachment element or portion thereof that is opposite the first edge. In certain embodiments, the width of an attachment element or portion thereof is a distance from a third edge of an attachment element or portion thereof to a fourth edge of the attachment elements or portion thereof that is opposite the third edge and may be measured in a direction transverse to the length of the attachment element or portion thereof. Likewise, the height of an attachment element or portion thereof may be the vertical height and/or a distance from a fifth edge of an attachment element or portion thereof to a sixth edge of the attachment element or portion thereof that is opposite the fifth edge and may be measured in a direction transverse to the length and width of the attachment element or portion thereof. In some aspects, the length of an attachment element or portion thereof is longer than its width. In some aspects, the length of an attachment element or portion thereof is equal to its width. In some aspects, the length of an attachment element or portion thereof, e.g., a sheet, may range from 5 cm to 100 cm, such as from 10 cm to 50 cm, such as from 20 cm to 35 cm. In addition, the width of an attachment element or portion thereof may range from 5 cm to 100 cm, such as from 10 cm to 50 cm, such as from 20 cm to 35 cm. Furthermore, the height of an attachment element or portion thereof may range from 0.1 cm to 5 cm, such as from 0.1 cm to 3 cm, such as from 0.1 cm to 1 cm.

In addition, a sheet of an attachment element may also be shaped as a pouch having an opening for receiving a portion of a mammal therein. In certain aspects, sheets are shaped substantially like a glove, a mitten, or a sock. In some aspects, sheets have a continuous exterior wall enveloping an interior cavity and an opening sized and shaped to receive a portion of a mammal, e.g., a hand and/or foot.

A sheet, in some aspects, also has the requisite strength to be applied according to the subject methods without tearing or loosening from a portion of a mammal. In some embodiments, a sheet is elastic, e.g., biased to retain an initial configuration but is stretchable from that configuration. In other embodiments, a sheet is non-elastic. A sheet may also be composed of a material such that it does not cause irritation, e.g., skin irritation, on a contacting portion of a mammal.

In some embodiments, one or more sheets of attachment elements are operatively connected to a container and/or and inflow element and/or an outflow element of a device. In certain aspects, sheets are attached to a container fixedly, e.g., by adhesive, and/or by a sewn seam, and/or by a melted seam and/or by one or more, e.g., two, attachment elements positioned therebetween. In some embodiments, sheets are a continuous body, e.g., body of material, with and/or composed of the same material as a container or a portion of a container of a device. In some embodiments sheets are removably attached to a container. In such embodiments, sheets may be snapedly connected to a container and/or connected by friction fit and/or a zipper and/or one or more buttons and/or one or more clips, e.g., clips configured to mate.

In various aspects of the subject devices, one or more surfaces of a container are attached to an attachment element or portion thereof, e.g., a sheet. The surface attached to the container may be opposite, e.g., on an opposite side of a container than, a surface contacting a heat exchange surface of a mammal during use. In some embodiments, an attachment element or portion thereof, e.g., a sheet, is sized and/or shaped to envelop or substantially envelop a container and/or a portion of a mammal, e.g., a hand and/or foot. In some embodiments, the area of a surface an attachment element or portion thereof, e.g., a sheet, contacting a surface of a container is larger than the surface area of the surface of the container which the attachment element is contacting.

In some embodiments, one or more, e.g., two, three, four, etc., edges, e.g., a peripheral edge of a container, align with one or more, e.g., two, three, four, etc., peripheral edges of an attachment element or portion thereof, e.g., a sheet. In some embodiments of the subject devices, an attachment element or portion thereof, e.g., a sheet, is sized and/or shaped to form an exterior layer of the device during use, and the container is sized and/or shaped and positioned with respect to other portions of the device to form an interior layer of the device, e.g., a layer configured to contact a heat exchange surface of a mammal.

Attachment elements may also include one or more, e.g., two, three, four, five, etc., attaching bands, e.g., straps. In various embodiments, attachment elements, or portions thereof, e.g., attaching bands, are configured to secure a heat exchange element or a portion thereof, e.g., a container to a portion of a mammal by forming an opening between the attachment element and, the heat exchange element or portion thereof, e.g., the container. Such an opening may be formed on a first side by the attachment element and formed on a second side which is opposite the first side by the heat exchange element or portion thereof, e.g., the container. Such an opening may also be configured to receive, e.g., slidably receive, a portion of a mammal, e.g., a portion of a hand or foot, therethrough. Additionally, an opening in a device may be sized and shaped such that a fit, e.g., a friction fit, e.g., a friction fit which is comfortable to the mammal, is obtained between the portion of the mammal and the device or portion thereof, e.g., container, wherein the fit may affix the portion of the mammal to the container. An opening formed between an attachment element and a heat exchange element may be sized and/or shaped to have a minimal impact, e.g., no impact, on the circulation of blood through a portion of the mammal inserted therein and/or therethrough. Furthermore, an attachment element or portion thereof, e.g., a sheet and/or attaching band, composed of an elastic material, may be biased to retain a portion of a mammal, e.g., a hand or foot, between an attachment element and a container, e.g., within an opening in a device. An attachment element or portion thereof, e.g., a sheet and/or attaching bands, may also be biased to retain, e.g., retain by a friction fit, a portion of a mammal within, e.g., between two or more portions of, a device.

Attaching bands of attachment elements may have any suitable size and/or shape. For example, an attaching band may be substantially planar and have a peripheral shape defining or substantially defining a rectangle, square, oval, or other shape. In certain embodiments, attaching bands are rectangular and have a length, width and height. In various aspects, the length and width of attaching bands are both many times e.g., 5 or more, 10 or more, 20 or more, or 50 or more times, longer than the height of an attaching band.

In some embodiments, attaching bands have a first end and a second end and are affixed at the first and second end to the container of a device. In some aspects, a first end of an attaching band is affixed to or proximate a first edge of a container and/or a second end of an attaching band is affixed to or proximate a second edge of a container, and/or wherein the second edge of a container is on an opposite side of the container than the first edge. In certain aspects, attachment elements or portions thereof, e.g., attaching bands, are attached to a container fixedly, e.g., by adhesive, and/or by a sewn seam, and/or by a melted seam. In some embodiments, attachment elements are a continuous body, e.g., body of material, with and/or composed of the same material as a container or a portion of a container of a device.

In certain embodiments, attachment elements or portions thereof, such as attaching bands and/or sheets, have more than one portion, e.g., two portions, which are removably and repeatedly attachable to one another. Such portions may be attachable to one another by a connection formed, for example, by a hook and loop (i.e., Velcro™) fastener, and/or one or more snaps, and/or buttons, and/or holes, and/or hooks, and/or clips, such as clips or buckles, e.g., plastic or metal clips or buckles, having a reciprocating male and female portion. In some embodiments, attachment elements have a number of fasteners ranging from 1 to 100, such as from 1 to 10, such as from 1 to 5. In some aspects, attachment elements include five or fewer, or four or fewer fasteners. Certain variation also include a single fastener or two fasteners on a first portion, e.g., a first portion as described above, of an attachment element and a number of fasteners ranging from 1 to 5, such as 3 on a second portion of an attachment element.

Attachment elements or portions thereof, such as sheets and/or attaching bands, may be composed of any suitable material. For example, attachment elements or portions thereof may be composed of one or more fabrics, e.g., cotton and/or polyester and/or nylon. Attachment elements or portions thereof may also be entirely or partially composed of one or more elastic materials. Elastic materials are materials which, for example, are biased to retain their initial un-biased shape when one or more forces are exerted thereon which cause the materials to assume, e.g., to temporarily assume, a second “biased” shape. Various aspects of attachment elements, such as attachment elements or portions thereof having elastic materials are configured to temporarily assume a second “biased” shape when a portion of a mammal is inserted into and/or retained within a device, such as between a container and an attachment element. Embodiments of elastic materials are materials which are configured to exert, when retained in a “biased” shape, a force, a sufficient force, on a portion of a mammal and/or one or more portions of a container such that the surface area between the container and a portion of a mammal, such as a heat exchange surface, is maximized. Furthermore, attachment elements or portions thereof, e.g., attaching bands and/or sheets, and/or elastic materials of attachment elements, may be composed of one or more polymeric materials, e.g., a single polymeric material. Specific polymeric materials of interest include, but are not limited to: plastics, rubbers, silicones, etc.

Materials of which attachment elements may be composed also include materials which have the requisite strength for the disclosed systems, devices and methods to effectively be employed. For example, materials of which attachment elements or portions thereof, such as sheets and/or attaching bands, are composed include materials which are resistant to degrading and/or tearing and/or breaking when attachment elements are repeatedly employed to secure a container to a portion of a mammal, such as a heat exchange surface. Materials of which attachment elements or portions thereof may be composed also include materials which are sterilizable and/or re-sterilizable. Materials of which attachment elements are composed, in various embodiments, also include materials which are disposable, e.g., disposable after a single use, such as biodegradable and/or organic materials.

In various aspects of the subject devices, the devices include one or more pads, such as a pad which is operatively connected e.g., attachable to or detachable from the remaining portions of the device e.g., the heat exchange element or portion thereof, e.g., the container and/or the attachment element or portion thereof e.g., a sheet. Aspects of pads of the disclosed devices may be configured to enhance a subject mammal's comfort, e.g., comfort relating touch and/or temperature, during employment of the subject systems, device and methods. Various aspects of pads of the disclosed devices are configured to increase the effectiveness of the subject systems, device and methods, for example, by assisting to maintain a temperature of or immediately surrounding a fluid within a container during operation of the disclosed systems and devices. Embodiments of pads of devices are sized and/or shaped to fit, e.g., comfortably fit, against and/or around a portion of a mammal, e.g., a palm and/or a sole. Certain embodiments of pads of devices are sized and/or shaped to fit within, e.g. entirely within, other portions of a device, e.g., an attachment element or portion thereof, such as a sheet.

In some embodiments, the subject thermoregulatory devices are configured to be portable. For example, devices may be sized and shaped to be easily moved from one location to another by an amount of force capable of being exerted by an average child and/or adult human arm and/or hand. In certain aspects, the negative pressure devices described herein have a mass ranging, for example, from 10 g to 3000 g, from 100 g to 2900 g, from 500 g to 2000 g, or from 1300 g to 1400 g.

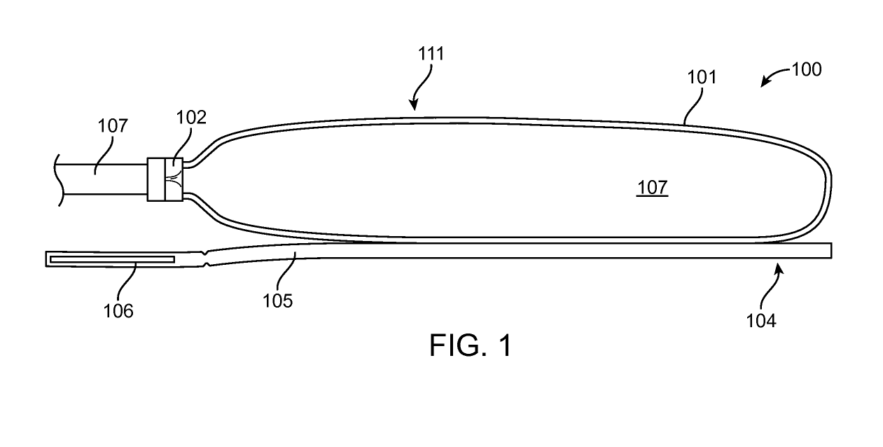

FIG. 1 depicts one embodiment of a thermoregulatory device 100 which can be utilized, e.g., within the subject systems, in accordance with the subject methods. More specifically, FIG. 1 shows a thermoregulatory device 100 having a heat exchange element 111 including a container 101 and an outflow element 102 coupled to a fluid conveying element, e.g., a tube 107. Also shown in FIG. 1 is an attachment element 104 operatively coupled to a surface of the container 101 of the heat exchange element 111. The attachment element 104 includes a sheet 105 having a fastener 106 thereon.

FIG. 2 depicts the embodiment of the thermoregulatory device 100 in use. FIG. 2 specifically illustrates the container 101 of the heat exchange element 111 of the device 100 stably associated with a heat exchange surface 109 of a portion of a mammal, e.g., a hand 108. In FIG. 2, the sheet 105 of the attachment element 104 of the device 100 is shown wrapped around and enveloping the wrist 110 and hand 108 of the mammal.

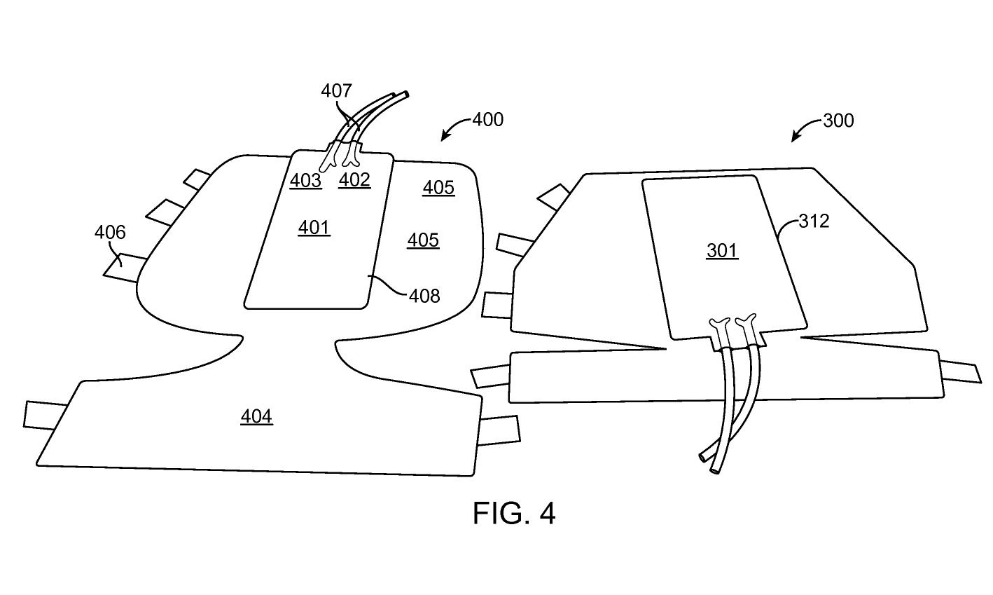

An embodiment of a device 300, configured to wrap around a portion of limb of a mammal, e.g., a hand, is shown, for example, in FIGS. 3 and 4. More specifically, device 300 includes a heat exchange element 312 including a container 301 as well as an inflow element 303 and an outflow element 302. Inflow element 303 and outflow element are both connected to fluid conveying elements, e.g., tubes 307. In addition, the inflow element 303 and outflow element 302, as shown, as well as the inflow and outflow elements shown in FIGS. 1, 2, and 4, are interchangeable, meaning that each may operate as either an inflow or outflow element at a given time during use. FIG. 3 also illustrates an attachment element 304 having a sheet 305 and fasteners 306.

Another embodiment of a device, configured to wrap around a portion of limb of a mammal, e.g., a foot, is shown, for example, in FIG. 4. Device 400 specifically includes a heat exchange element 408 having a container 401, as well as an inflow element 403 and outflow element 404 operatively connected to fluid conveying elements, e.g., tubes 407. Also provided in FIG. 4 is an attachment element 404, e.g., an attachment element configured to wrap around a portion of a limb of a mammal, e.g., a foot. The attachment element 404 includes sheet 405 and fasteners 406.

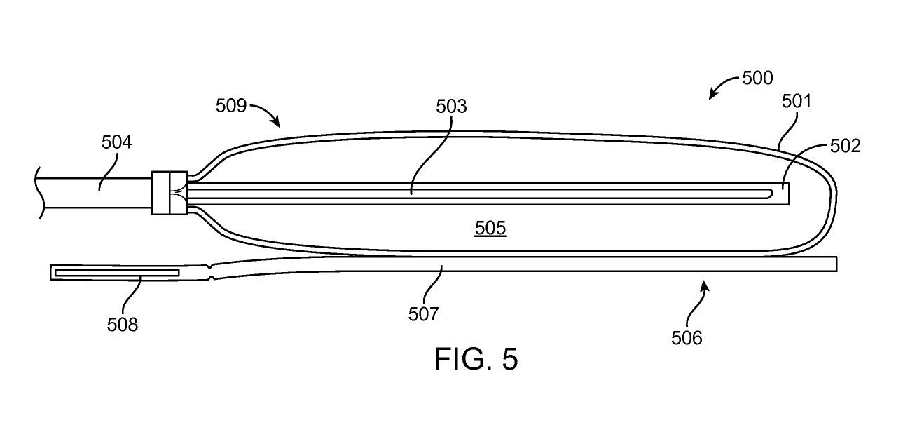

In addition, FIG. 5 depicts one embodiment of a thermoelectric device 700 which can be utilized, e.g., within the subject systems, in accordance with the subject methods. More specifically, FIG. 5 shows an electrical thermoregulatory device 500 including a heat exchange element 509 having a container 501 and an electrical heater and/or cooler 502 having an electrical heating and/or cooling component 503 therein. The electrical heater and/or cooler 502 having an electrical heating and/or cooling component 503 is operatively connected to electrical cord 504. Electrical cord 504 may, in turn provide electrical connections to a power source and/or a controller and/or an on/off switch, as such elements are described herein. FIG. 5 also illustrates a holder e.g., a holding pad 505 configured to secure the electrical heating and/or cooling component 503 within the container 501. Also shown in FIG. 5 is an attachment element 506 operatively coupled to a surface of the container 501. The attachment element 506 includes a sheet 507 having a fastener 508 thereon.

In some instances, the devices may be incorporated into a suit or analogous article configured to enclose a portion of, if not all of, a mammal, e.g., a personal protective suit, e.g., a chemical or biohazard protective suit (e.g., DuPont™ Tychem® QC and DuPont™ Tychem® SL biohazard suits worn by ebola workers) or analogous structure.

### Systems

Aspects of the invention further include systems that include one or more thermoregulatory devices, e.g., as described above. Aspects of the subject systems may include one or more, e.g., two, three, four, five, etc., thermoregulatory devices, such as any of the devices described herein. In some versions, and as noted above, the thermoregulatory devices are devices for transferring heat with a portion of a mammal having a compromised thermoregulatory system and include one or more, e.g., two, three, four, etc., heat exchange elements. Heat exchange elements of the subject devices may be heating and/or cooling elements, and may be electrical or fluid devices, e.g., as described above.

The disclosed systems, in various embodiments, also include one or more power sources. A power source may, in some aspects, be one or more batteries, e.g., a portable and/or self-contained battery, an electrical outlet, or another source of electrical power, e.g., an electrical generator, e.g., a portable electric generator. In some embodiments, a power source may be operatively connected to one or more switches, e.g., a manual switch, configured to start and/or stop administration of power e.g., electrical power to one or more elements of the subject systems, e.g., an electrical heater and/or cooler, and/or a pump, such as a circulating pump, from the power source and/or to thereby turn one or more elements of the subject systems on and/or off. In some embodiments, a power source is operatively connected to a heat exchange element of the subject devices. In some embodiments, power sources provide power to a heat exchange element including an electrical heater and/or cooler.

In various embodiments, the systems, including each of the components thereof, are configured for surgical operating room use. As such, a system may be configured to transfer heat with, e.g., transfer heat to and/or from, a heat exchange surface of a mammal during a surgical procedure on the mammal. Additionally, in some instances, the systems are configured for intra-operative use.

The subject systems, in various embodiments, include one or more circuits, e.g., a fluidic circuit, such as a loop, e.g., a sealed and/or closed loop, through which fluid, e.g., water, may circulate and/or recirculate and/or be retained. Such a circuit may include a thermoregulatory device and temperature modulator and may also include any of the other elements of the systems described herein, as well as tubing extending between and/or operatively connecting each of the elements in the circuit. A circuit of the subject systems may also include or be configured to provide, e.g., only provide, a unidirectional fluid flow therethrough. Additionally, the elements of the systems described herein may be placed in any order within a circuit that allows the systems to function in the manner and for the purposes described herein.

The systems, in some embodiments include one or more valves, e.g., fluidic valves, positioned within one or more circuits, e.g., a fluidic circuit, of the systems. The valves may be actuable and may be configured to start and/or stop fluid flow through the valve and/or through a circuit when they are actuated. The valves may also be automatic, e.g., electronic, and/or manual.

In some embodiments, the systems include one or more e.g., two, three, four, five, etc., temperature modulators, e.g., a temperature modulator operatively connected to an inflow element of a thermoregulatory device. Thermoregulatory devices and temperature modulators of the subject systems are distinct components that are configured to work together, for example, to transfer heat from a portion of a mammal.

In various embodiments, temperature modulators of the disclosed systems are configured to heat and/or cool and/or hold fluid, e.g., fluid as described above which the thermoregulatory device, or a portion thereof, e.g., a heat exchange element, is configured to receive therethrough. In other words, a temperature modulator may be configured to transfer energy, e.g., thermal energy, e.g., heat, with the fluid. As such, in some aspects, a temperature modulator is configured to adjust, e.g., increase and/or decrease, a temperature of a fluid within, e.g., flowing through, the temperature modulator. Accordingly, the subject temperature modulators may be configured to increase and/or decrease and/or maintain a temperature of a fluid, e.g., a fluid within the system, which is necessary to heat and/or cool the body core temperature of a mammal when the system is in operation.

Additionally, in some embodiments, a temperature modulator includes a source of heating fluid and/or cooling fluid, as such terms are described above. In various aspects, temperature modulators include a heater, e.g., an electrical heater, and/or a cooler, e.g., an electrical cooler and/or a heat sink. Such a heater may be configured to heat fluid flowing through the temperature modulator and such a cooler may be configured to cool fluid flowing through the temperature modulator. Heaters, in various embodiments, are configured to maintain a temperature, e.g., a temperature in one or more internal compartments thereof, in a temperature range, for example, from 30° C. to 60° C., from 30° C. to 50° C., or from 35° C. to 45° C. and/or are thereby able to heat fluid retained therein or flowing therethrough to a temperature within the same ranges. Coolers, in various embodiments, are configured to maintain a temperature, e.g., a temperature in one or more internal compartments thereof, in a temperature range, for example, from 1° C. to 35° C., from 10° C. to 30° C., from 10° C. to 25° C., from 10° C. to 22° C., or from 12° C. to 19° C. and/or are thereby able to cool fluid retained therein or flowing therethrough to a temperature within the same ranges. Temperature modulators, in various aspects, are configured to heat and/or cool the fluids described herein when the fluid is placed in thermal conductivity e.g., in a physical relationship such that heat passes between the temperature modulator and the fluid, with the temperature modulators.

In some embodiments, temperature modulators have one or more, e.g., two, three, four, etc., cavities therein. In various embodiments, a cavity is configured to receive a flow of fluid therethrough and/or retain fluid therein for a period of time. Accordingly, in some embodiments, the subject cavities include one or more cavities, e.g., a pad, through which fluid, e.g., water, may perfuse. In some embodiments, the cavities include one or more containers, such as containers having any of the characteristics described in association with the thermoregulatory devices above. Additionally, in some embodiments, the temperature modulators include one or more water perfusion pads.

In some aspects, a temperature modulator is operatively connected to a heat exchange element, or a portion thereof, e.g., an inflow element and/or outflow element of a thermoregulatory device. Such an operative connection may be a sealed connection and allow fluid to flow, e.g., flow within a circuit, between the temperature modulator and thermoregulatory device. Likewise, in some aspects, the temperature modulator may be operatively connected to one or more fluid conveying elements, e.g., tubes, which are in turn operatively connected to an inflow element and/or outflow element of a thermoregulatory device.

In some aspects, temperature modulators include a control unit, e.g., an electrical control unit, configured for controlling heating and/or cooling fluid within or flowing through the temperature modulator. In some embodiments, control units of temperature modulators have one or more switches, e.g., a switch for manually adjusting the temperature of fluid flowing through the temperature modulator. In some embodiments, control units of temperature modulators are configured to automatically adjust the temperature of, e.g., heat and/or cool, fluid flowing through the modulators. In various instances, control units include a central processing unit and/or other elements of a computer, e.g., a power source connector configured to operatively connect the control unit or element thereof to a power source.

In various embodiments, the subject systems, and/or the components thereof, include one or more, e.g., two, three, four, five, etc., temperature monitors, e.g., thermometers, e.g., implantable thermometers, configured for measuring a body core temperature of a mammal. In some embodiments, control units of temperature modulators are configured to automatically adjust the temperature of fluid flowing through the modulators, and/or an electrical heater and/or cooler, based on the body core temperature of a mammal. As such, in some embodiments, temperature modulators and/or control units thereof are operatively connected, e.g., electrically connected, to one or more temperature monitors configured for measuring a body core temperature of a mammal.

In some embodiments, the subject systems include one or more, e.g., two, three, four, five, etc., pumps, e.g., fluidic pumps. In certain aspects, a pump may be operatively connected to a thermoregulatory device, or components thereof, such as a heat exchange element, or a portion thereof, e.g., an inflow element and/or an outflow element, and/or a fluid conveying element, e.g., one or more tubes, and/or a temperature modulator and/or to other elements of the disclosed systems. Pumps may also be configured to circulate and/or recirculate, e.g., draw, fluid through a circuit, e.g., a fluidic circuit, of the disclosed systems as well as the pumps and/or other disclosed elements of the systems, e.g., thermoregulatory device and/or temperature modulator. In some aspects, pumps are unidirectional and/or are configured to suck fluid in to a first opening in the pump and propel fluid out of a second opening of a pump. In some variations, pumps are vacuum pumps and/or pressurizing pumps and/or centrifugal pumps. In some embodiments, pumps are configured to exert pressure on fluid and thereby pull fluid, e.g., heating fluid and/or cooling fluid, e.g., water, into the pump and/or push fluid out of the pump. In some embodiments, pumps, e.g., circulating pumps are operatively connected to a power source, e.g., a battery and/or a switch, e.g., a switch configured to turn the pump on and/or off, such as a manual switch.

In some embodiments, the subject systems may include one or more filters, e.g., a fluidic filter, e.g., a fluidic filter positioned in a fluidic circuit. The filter may be configured to remove one or more impurities from a fluid flowing therethrough. Also, the disclosed systems may include one or more bubble traps. A bubble trap may be configured to trap and/or remove bubbles from fluid e.g., fluid circulating within one or more elements of the disclosed systems, e.g., fluid circulating in a fluidic circuit. In some embodiments, a filter and/or bubble trap of the disclosed systems is operatively coupled to one or more elements of the disclosed systems, e.g., a thermoregulatory device and/or tubing and/or a pump. In some embodiments, a filter and/or bubble trap is positioned in a circuit, e.g., a fluidic circuit, between a thermoregulatory device and/or tubing and/or a pump. In some embodiments, a filter and/or bubble trap is operatively coupled to an outflow element of a thermoregulatory device and/or tubing operatively coupled to the outflow element.

In various aspects a filter and/or bubble trap and/or another element, e.g., tubing, of the disclosed systems may be operatively coupled, e.g., fluidically coupled, to one or more ports, e.g., a priming port. In some aspects, a port may be a fluidic port and may be configured to receive one or more liquids, e.g., water, and/or gas therethrough. In some embodiments a priming port may be a port through which fluid may pass in and/or out of the disclosed systems. In some embodiments, bubble traps of the disclosed systems are configured to remove bubbles from fluid injected into a priming port before the fluid is released to other elements of the disclosed systems, e.g., injected into a circuit, e.g., a fluidic circuit of the disclosed systems. In some embodiments, the systems are sealed, e.g., fluidically sealed, but for an openable and/or closable opening in a priming port of the systems.

The subject systems also, in various instances, include one or more valves therein. Such valves may be configured to allow fluid therethrough and/or prevent fluid from passing therethrough. The valves may be manual and/or automatic valves and may be electrical, e.g., electrically controlled, valves. In some embodiments, valves are positioned within a circuit, e.g., a fluidic circuit, of the disclosed systems between two or more other elements of the systems, such as between a pump and/or a temperature modulator and a thermoregulatory device or portion thereof, e.g. an inflow element. In some embodiments, the valves are thermostatic mixing valves, e.g., valves configured to mix one or more fluids therein to produce an outflow of fluid having a uniform temperature, e.g., a desired temperature. In some embodiments, valves, e.g., thermostatic mixing valves, are configured to receive a flow of fluid therethrough. In some embodiments, valves, e.g., thermostatic mixing valves, include two or more inputs, each of which are configured to receive a flow, e.g., an inflow, of fluid at a different temperature and one or more outputs, which are configured to receive a flow, e.g., an outflow, of fluid at a uniform temperature therethrough. In some embodiments, valves, e.g., thermostatic mixing valves, include one or more containers and/or one or more mixing components therein configured to mix one or more fluids to produce a fluid having a single uniform temperature. In some embodiments, valves, e.g., thermostatic mixing valves, are configured to deliver a fluid having a single uniform temperature to one or more other elements of the subject systems, e.g., a thermoregulatory device or a portion thereof, e.g., an inflow element.

In some variations, the subject systems or portions thereof, e.g., a circuit, e.g., a fluidic circuit, include one or more (e.g., two, three, four, five or more, etc.) fluid conveying elements, e.g., tubes, e.g., tubes configured to receive a fluid therethrough. In various embodiments, the one or more fluid conveying elements, e.g., tubes, may include or have any characteristics of the tubes described above in association with the subject devices and/or are configured, e.g., sized and/or shaped to perform according to any of the subject methods. In some embodiments, the fluid conveying elements, e.g., tubes, are flexible and/or rigid and/or polymeric. In some aspects, the fluid conveying elements, may be operatively connected e.g., sealably and/or adhesively connected, to one or more elements of the disclosed systems. In various aspects, the systems include one or more fluid conveying elements, e.g., tubes, between some or all of the elements of the systems. For example, the subject systems may include one or more fluid conveying elements, between and/or operatively connected to a thermoregulatory device and/or portions thereof, e.g., an inflow and/or outflow element; and/or a bubble trap; and/or a circulating pump; and/or a temperature modulator and/or a valve, e.g., a thermostatic mixing valve; and/or the thermoregulatory device.

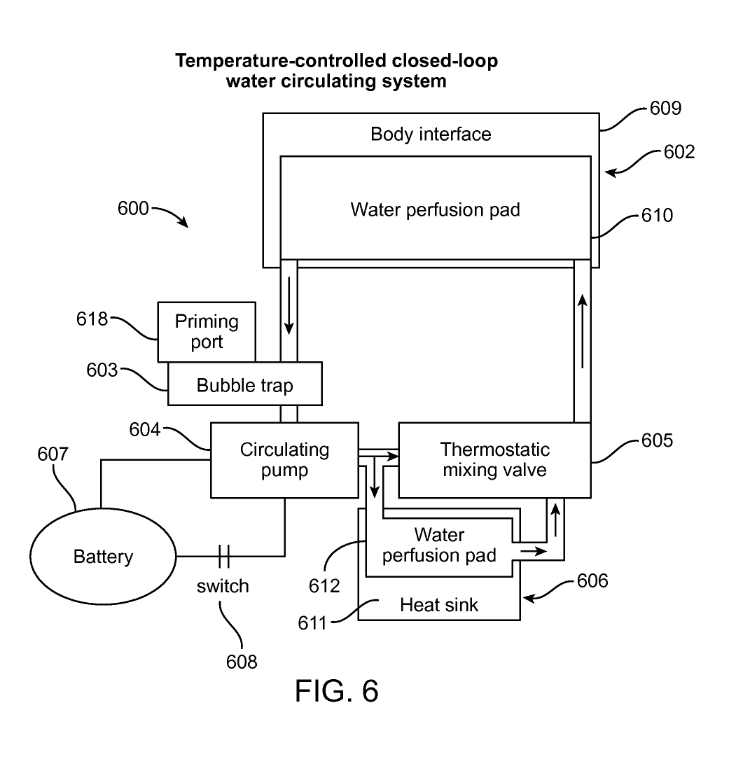

One embodiment of the subject systems is shown schematically in FIG. 6. FIG. 6 specifically illustrates a system 600 having a fluid flow, e.g., a unidirectional fluid flow, therethrough. FIG. 6 provides a fluidic circuit configured such that fluid may pass in sequence through a series of operatively connecting elements. More specifically, fluid is configured to pass out of a thermoregulatory device 602, through a bubble trap 603, through a circulating pump 604, through a thermostatic mixing valve 605 and/or thorough a temperature modulator 606, and then through the thermostatic mixing valve 605, and then back into the thermoregulatory device 602. In the embodiment of the system shown, the circulating pump is operatively connected to a battery 607 and a switch 608. In addition, the thermoregulatory device 602 includes a body interface 609 as well as a water perfusion pad 610. Furthermore, the temperature modulator 606 includes a water perfusion pad 612 and a cooler, e.g., a heat sink 611. The bubble trap 603 is also operatively coupled to a priming port 618.

In some instances, the systems may be incorporated into a suit or analogous article configured to enclose a portion of, if not all of, a mammal, e.g., a biohazard suit or analogous structure.

### Utility

As demonstrated above, the subject methods, devices and systems are directed to transferring, e.g., removing or introducing, heat with a portion of a mammal. As such, the subject methods are suitable for use in a variety of different applications, where particular applications include the control and/or maintenance of a mammal's body temperature, e.g., core body temperature, during a surgery on a mammal, and/or treatment of normal and abnormal physiological conditions, e.g., disease and/or discomfort (e.g., when wearing a thermally compromising suit, such as a biohazard suit, where core body heat extraction and/or infusion is desirable. As such, the methods, devices and systems described herein find use in a variety of different applications. Applications of interest include, but are not limited to: 1) Forestalling symptom exacerbation in individuals with multiple sclerosis who are sensitive to temperature; 2) Maintaining normal internal body temperatures (normothermia) in individuals rendered thermally defenseless by anesthetic agents; 3) Restoring normothermia in individuals during emergence from anesthesia; 4) Inducing changes in regional distribution of cardiac output to reduce central blood pressure and relieve symptoms of a variety of acute medical conditions (e.g., migraines); 5) Facilitating recovery from heat and cold stress; 6) Increasing physical exercise capacity in thermally hostile environments, e.g., biohazard or analogous suits; 7) Reducing or eliminating post exercise muscle soreness; 8) Reducing the long term neuronal damage from a traumatic head injury, or a hypoxic/ischemic insult; 9) Treatment of thermal illnesses; 10) Mitigating the duration and severity of hot flash symptoms, etc. By treatment is meant both a prevention and/or at least an amelioration of the symptoms associated with the condition afflicting the subject is achieved, where amelioration is used in a broad sense to refer to at least a reduction in the magnitude of a parameter, e.g., symptom, associated with the condition being treated. As such, treatment also includes situations where the condition, or at least symptoms associated therewith, are completely inhibited, e.g. prevented from happening, or stopped, e.g., terminated, such that the subject no longer suffers from the condition, or at least the symptoms that characterize the condition.

Applications of interest include those characterized by reducing thermal stress in a mammal. By reducing thermal stress is meant lowering or ameliorating a physiological strain experienced by a mammal resulting from change in the mammal's thermoregulatory system (e.g., in response to external or internal stimulus), as compared to a suitable control in which the subject methods are not employed. The reduction in thermal stress may be manifested in a number of different ways, e.g., as maintenance of normothermia, etc., such as described in greater detail below.

One application in which the subject systems, devices and methods find use in providing normal internal body temperatures i.e., normothermia, e.g., normal core body temperatures, intra-operatively. Providing normal internal body temperatures may include, for example, maintaining a core body temperature of a mammal at a temperature of ranging from 35 to 38, such as 35.25 to 37.5, including 35.5 to 37.25° C., e.g., maintaining temperature at 37° C. and/or within 2° C. and/or within 1.5° C. of 37° C., during a give procedure. In some instances, normothermia is achieved with the subject methods and devices and system by limiting any core body temperature reduction during a surgical procedure to 1.75° C. or less, such as 1.5° C. or less, including 1.25° C. or less, such as 1° C. or less, e.g., 0.75° C. or less, including 0.5° C. or less, e.g., 0.25 or less, including no drop in core body temperature, during an extended period of anesthesia, e.g., 1 hour or longer, such as 2 hours or longer, e.g., 3 hours or longer, where in some instances the extended period of anesthesia is 12 hours or less, such as 9 hours or less, e.g., 6 hours or less, including 4 hours or less.

Particular applications in which the subject systems, devices and methods find use include providing a mammal with a safer and/or more comfortable surgical procedure and/or recovery from the procedure than the surgical procedure and/or recovery would be without the systems, devices and/or methods. For example, a surgical procedure and/or recovery associated with the subject systems, devices and/or methods may reduce a health risk e.g., a significant health risk, to the mammal undergoing the surgical procedure.

One way the subject systems, devices and methods find use include providing a mammal with a safer and/or more comfortable surgical procedure is to help maintain conditions, e.g., safe conditions, such as a natural temperature, of the internal organs, e.g., heart, lungs, liver, etc., of a mammal during the procedure. The subject systems, devices and methods also find use in providing a mammal with a safer and/or more comfortable surgical procedure by helping to monitor and/or maintain the mammal's intra-operative vital statistics, e.g., heart rate and/or breathing rate, at a safe and/or desired level.

In some circumstances, the subject systems, devices and methods reduce intra-operative health risk by helping to control a mammal's blood pressure as compared to what would occur in the absence of the systems, devices and/or methods, from a mammal to occur. Additionally, in various circumstances, the subject systems, devices and methods allow a mammal to have a reduced healing time and/or faster recovery from a surgical procedure.

In various embodiments, the subject systems and methods are applied for alleviation or treatment of dangerous intra-operative conditions. By dangerous conditions is meant conditions which pose or could pose a health risk, e.g., a significant health risk, such as a health risk which could have a permanent negative effect on the mammal's health, to a mammal during a surgery or during recovery from a surgery. Examples of dangerous conditions include a low and/or high body temperature and/or heart rate and/or breathing rate. Also, by treatment is meant at least an alleviation of one or more of the circumstances providing for the dangerous conditions.

Additionally, as noted above, the subject systems, devices and methods find use include providing a mammal with a comfortable surgical procedure and/or recovery. As such, relief and/or treatment provided by the subject systems, devices and methods may be related to making a mammal, e.g., a mammal undergoing or recovering from a surgical procedure, more comfortable, such as less scared or agitated.

In some aspects, mammals may utilize the systems, devices and methods described herein in a preemptive manner. For example, a mammal expecting to undergo an intra-operative drop in core body temperature may apply the, systems, devices and/or methods to forestall or prevent such a drop in temperature.

### Kits

Also provided are kits that at least include one or more thermoregulatory devices, e.g., as described above, and which may be used according to the subject methods. The subject kits may include two or more, e.g., a plurality, three, four, five, eight, ten, etc., thermoregulatory devices or other system components according to any of the embodiments described herein, or any combinations thereof. Kits may also include packaging, e.g., packaging for shipping the systems and/or devices without breaking.

In some embodiments, the kits include a set of two or more, e.g., four or more, thermoregulatory devices for transferring heat with a portion of a mammal, e.g., a mammal having a compromised thermoregulatory system. Each device of a set may include a heat exchange element. In some embodiments, heat exchange elements include a container configured to contact only a portion of a limb of a mammal, and/or an inflow element operatively connected to the container, and/or an outflow element operatively connected to the container. In various aspects, heat exchange elements include a heater and/or cooler, e.g., an electrical heater and/or cooler.

Various aspects of the subject kits may also include two or more devices, e.g., four devices, each of which include an attachment element, e.g., an attachment element configured to secure a container of a device to a heat exchange surface of a mammal. Such attachment elements may also include one or more sheets, such as a sheet configured to secure a container to a hand of a mammal and/or a sheet configured to secure a container to a foot of a mammal. In some embodiments of the kits, e.g., kits including four or more thermoregulatory devices, attachment elements of two or more of the devices include a sheet configured to secure a heat exchange element or portion thereof, e.g., a container, to the hand of a mammal and two or more of the devices include a sheet configured to secure a heat exchange element or portion thereof, e.g., a container to the foot of a mammal.

The subject kits, in some embodiments, include one or more fluid conveying elements, e.g., tubing, such as one or more tubes. Such elements may be any of the tubes described herein, e.g., tubes within or making up a circuit, e.g., a fluidic circuit. Such elements may also be configured to operatively connect to inflow and/or outflow elements of thermoregulatory device and/or other components of the subject systems.

In certain embodiments, the kits which are disclosed herein include instructions, such as instructions for using the subject devices and/or systems. The instructions are, in some aspects, recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging etc.). In other embodiments, the instructions are present as an electronic storage data file present on a suitable computer readable storage medium, e.g., Portable Flash drive, CD-ROM, diskette, etc. The instructions may take any form, including complete instructions for how to use the systems or devices or as a website address with which instructions posted on the world wide web may be accessed.

The following example is offered by way of illustration and not by way of limitation.

### Experimental

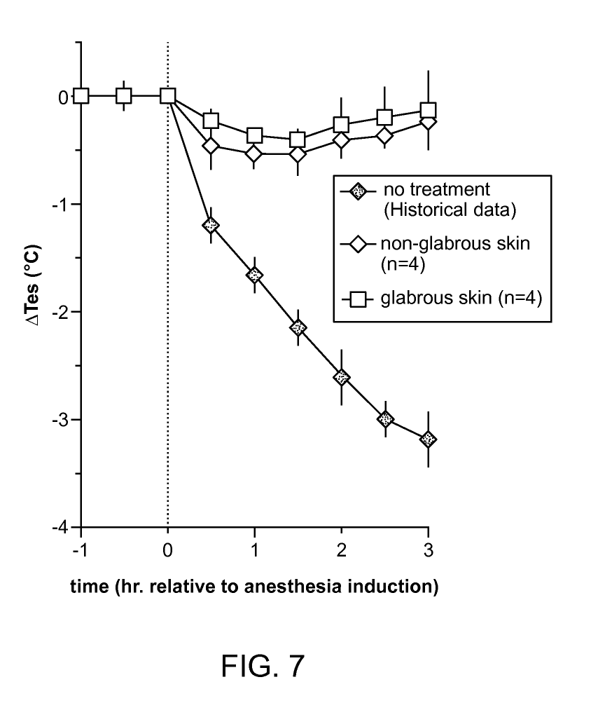

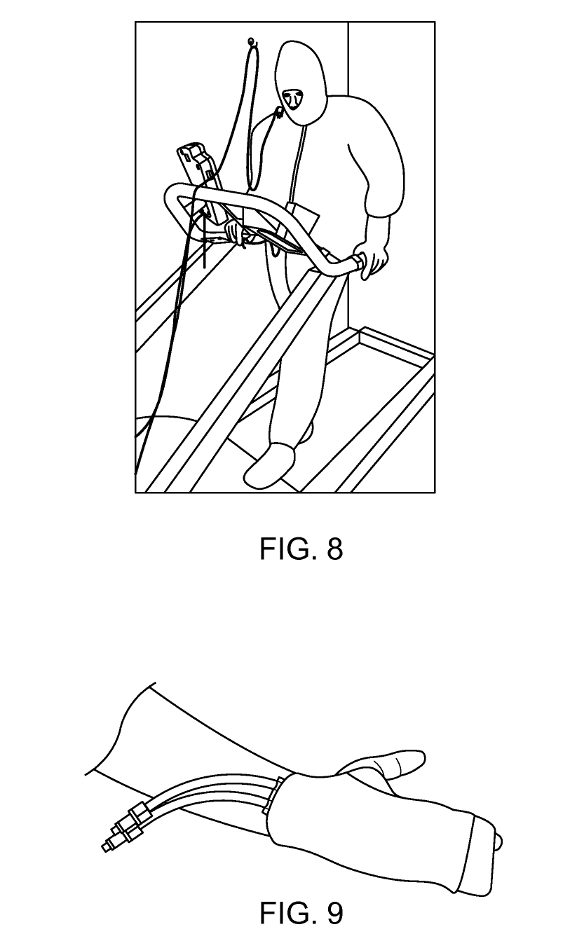

I. 8 subjects undergoing Transjugular intrahepatic portosystemic shunt (TIPS) surgery participated in this study. The duration of these surgeries are 3-4 hours during which the anesthetized subjects loose heat in a predictable manner. Without some sort of thermal intervention, core temperature of an anesthetized individual will drop at a rate of approximately 1° C./hr. The standard of practice for preventing the fall in core temperature during prolonged bouts of anesthesia is the use of forced air warming devices that deliver a temperature controlled stream of air to the general body surface; generally, the torso and legs. An alternative strategy, to deliver heat via a water circulating pad to the non-hairy skin regions of the hands and feet was compared to the standard of practice. The subjects were anesthetized following a standard practice protocol involving induction of anesthesia using a benzodiazapine agent (e.g., Midazolam), intubation with a tracheal tube, and maintenance of a surgical anesthesia plane using volatile anesthetic agents (e.g., isoflourane). Upon intubation, the subjects were equipped with an esophageal temperature (Tes) probe. Core temperature data was collected along with other vital signs as part of the standard patient monitoring protocol and recorded electronically at 1 minute intervals. The temperatures were down loaded from the digital record at 5 minute intervals. No patient identifiers were transferred with the Tes data. The temperature management equipment was applied to the patients 5-10 min after the initial induction of anesthesia—after the patients were equipped with the vital sign monitoring equipment and positioned on the operating table. Either a forced air warming blanket (Bear hugger, Arizant Healthcare, St Paul, Minn.) was draped over the patient's torso and legs or water perfused were placed in direct apposition to the soles of the feet and palms of the hands and held in place with a felt over cover. The forced air interfaces were plugged into a temperature controlled forced air blower (42° C.) and activated. The water perfusion pads were plugged into a commercial circulating water bath (Blanketrolll, CSZ, Cincinnati, Ohio) that delivered a constant stream of 41° C. to the four pads. The temperature management systems were left in place for the duration of the surgical procedure. Upon termination of the surgical procedure and removal of the tracheal tube the temperature management equipment was removed from the patient. The time and temperature data collected in each of these trials was entered into a single excel spreadsheet where the files were grouped by treatment. Treatment group means and SDs were calculated for each 5 min interval and plotted against time. For simplicity of illustration and comparison with literature values, the experimental group data was sampled at 30 min intervals and replotted (FIG. 7). For comparison with the treatment results, no treatment data was reproduced from results reported in the literature Matsukawa T, Sessler D I, Sessler A M, Schroeder M, Ozaki M, Kurz A, Cheng C: Heat flow and distribution during induction of general anesthesia. ANESTHESIOLOGY 1995; 82:662-73. Under this set of conditions the two treatment effects on core temperature were statistically equivalent. However, heating via the glabrous skin trended to provide a greater core temperature benefit.
II. A subject clad in biohazard protective suit and a balaclava as pictured in FIG. 8 walked at 2 miles per hour on a treadmill in a hot room (41° C.) for 40 minutes with or without cool water prefusion over the palm of the hands using a device of the invention as shown in FIG. 9. The palmar heat exchange device pictured in FIG. 9 is part of the portable cooling system worn under a personal protective suit pictured in FIG. 8. The palmar interface is a water perfusion pad enclosed in a tube of 4-way stretch lycra fabric, and is a physical embodiment of the system schematically illustrated in FIG. 6 and configured to transfer heat from a mammal via a glabrous surface.

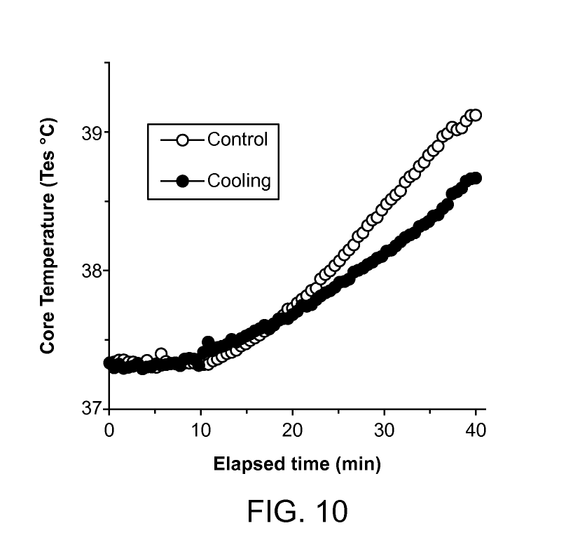

The results of the study are graphically illustrated in FIG. 10. As shown in FIG. 10, palm cooling using the device shown FIG. 9 dramatically reduced the rate of core temperature rise.

Notwithstanding the appended clauses, the disclosure is also defined by the following clauses:
1. A method of transcutaneous heat transfer, the method comprising: (a) stably associating a glabrous skin surface of a mammal with a thermoregulatory device comprising a heat exchange element and an attachment element, wherein the thermoregulatory device is configured to be associated with only a portion of a limb of a mammal; and (b) transferring heat through the glabrous skin surface without application of negative pressure in a manner sufficient to achieve transcutaneous heat transfer.
2. The method according to Clause 1, wherein the method comprises transferring heat from the heat exchange element of the thermoregulatory into the mammal through the glabrous skin surface.
3. The method according to Clause 1, wherein the method comprises transferring heat from the mammal into the thermoregulatory element of the device through the glabrous skin surface.
4. The method according to any of Clauses 1 to 3, wherein the heat exchange element is a fluid heat exchange element.
5. The method according to Clause 4, wherein the fluid heat exchange element comprises a temperature controlled liquid.
6. The method according to any of Clauses 1 to 3, wherein the heat exchange element is an electrical heat exchange element.
7. The method according to any of the preceding clauses, wherein vasoconstriction is compromised in the glabrous skin surface.
8. The method according to Clause 7, wherein the method further comprises compromising vasoconstriction in the glabrous skin surface.
9. The method according to Clause 8, wherein the method comprises producing anesthesia in the mammal.
10. The method according to any of the preceding clauses, wherein the glabrous skin surface comprises a palm.
11. The method according to any of Clauses 1 to 9, wherein the glabrous skin surface comprises a sole.
12. The method according to any of the preceding clauses, wherein the method comprises stably associating two or more glabrous skin surfaces of the mammal with a heat exchange element of a heat exchange device.
13. The method according to any of the preceding clauses, wherein the method is a method of maintaining reducing thermal stress in the mammal.
14. The method according to Clause 13, wherein the method is a method of maintaining normothermia of the mammal.
15. The method according to any of the preceding clauses, wherein the mammal is a human.
16. A thermoregulatory device configured to transfer heat through a glabrous surface of a mammal, the device comprising a heat exchange element and attachment element, wherein the thermoregulatory device is configured to be associated with only a portion of a limb of a mammal and does not include a negative pressure element.
17. The thermoregulatory device according to Clause 16, wherein the heat exchange element is a fluid heat exchange element.
18. The thermoregulatory device according to Clause 17, wherein the fluid heat exchange element comprises a flow path for a temperature controlled liquid.
19. The thermoregulatorydevice according to Clause 18, wherein the flow path comprises: (a) a high surface area heat transfer region; (b) an inflow element operatively connected to the heat transfer region; and (c) an outflow element operatively connected to the heat transfer region.
20. The thermoregulatory device according to Clause 16, wherein the heat exchange element is an electrical heat exchange element.
21. The thermoregulatory device according to any of Clauses 16 to 20, wherein the glabrous skin surface comprises a palm.
22. The thermoregulatory device according to any of Clauses 16 to 20, wherein the glabrous skin surface comprises a sole.
23. The thermoregulatory device according to any of Clauses 16 to 22, wherein the mammal is a human.
24. A system comprising: (a) a thermoregulatory device according to any of Clauses 16 to 23; and (b) a power source operatively coupled to the thermoregulatory device.
25. The system according to Clause 24, wherein the system further comprises a source of a temperature controlled liquid.
26. The system according to any of Clauses 24 and 25, wherein the system comprises two or more thermoregulatory devices according to any of Clauses 16 to 23.
27. A kit comprising: a set of two or more thermoregulatory devices according to any of Clauses 16 to 23.
28. The kit according to Clause 27, wherein the kit comprises two thermoregulatory devices configured to be associated with a human hand and two thermoregulatory devices configured to associated with a human foot.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of transcutaneous heat transfer, the method comprising:
(a) stably associating a glabrous skin surface of a mammal with a thermoregulatory device comprising a heat exchange element and an attachment element, wherein the thermoregulatory device is configured to be associated with only a portion of a limb of a mammal; and
(b) transferring heat through the glabrous skin surface without application of negative pressure in a manner sufficient to achieve transcutaneous heat transfer.

2. The method according to claim 1, wherein the heat exchange element is a fluid heat exchange element or an electrical heat exchange element.

3. The method according to claim 1 or 2, wherein vasoconstriction is compromised in the glabrous skin surface.

4. The method according to claim 3, wherein the method further comprises compromising vasoconstriction in the glabrous skin surface.

5. The method according to any of the preceding claims, wherein the method comprises producing anesthesia in the mammal.

6. The method according to any of the preceding claims, wherein the glabrous skin surface comprises a palm or a sole.

7. A thermoregulatory device configured to transfer heat through a glabrous surface of a mammal, the device comprising a heat exchange element and attachment element, wherein the thermoregulatory device is configured to be associated with only a portion of a limb of a mammal and does not include a negative pressure element.

8. The thermoregulatory device according to claim 7, wherein the heat exchange element is a fluid heat exchange element or an electrical heat exchange element.

9. The thermoregulatory device according to any of claim 7 or 8, wherein the glabrous skin surface comprises a palm a sole.

10. The thermoregulatory device according to any of the preceding claims, wherein the mammal is a human.

11. A system comprising:
(a) a thermoregulatory device according to any of claims 7 to 10; and
(b) a power source operatively coupled to the thermoregulatory device.

12. The system according to claim 11, wherein the system further comprises a source of a temperature controlled liquid.

13. The system according to any of claims 11 and 12, wherein the system comprises two or more thermoregulatory devices according to any of claims 7 to 10.

14. A kit comprising:
a set of two or more thermoregulatory devices according to any of claims 7 to 10.

15. The kit according to claim 14, wherein the kit comprises two thermoregulatory devices configured to be associated with a human hand and two thermoregulatory devices configured to associated with a human foot.
